(19) United States
(12) Patent Application Publication
(10) Pub. No.: US 2021/0380710 A1
(43) Pub. Date: Dec. 9, 2021

(54) A NOVEL ANTI-CD3/ANTI-CD20 BISPECIFIC ANTIBODY

(71) Applicant: Wuxi Biologics Ireland Limited, Dublin 1 (IE)

(72) Inventors: Yunying CHEN, Shanghai (CN); Qin MEI, Shanghai (CN); Zhuozhi WANG, Shanghai (CN); Jing LI, Shanghai (CN)

(21) Appl. No.: 17/057,607

(22) PCT Filed: May 29, 2019
(86) PCT No.: PCT/CN2019/089032

§ 371 Date: Nov. 20, 2020

(30) Foreign Application Priority Data
May 29, 2018 (CN) PCT/CN2018/088900

(51) Int. Cl. C07K 16/28; A61P 35/00

(52) U.S. Cl. CPC C07K 16/2887; C07K 16/2809; A61K 2039/505; C07K 2317/31; A61P 35/00

(57) ABSTRACT

Provided are a bispecific antibody comprising a first antigen-binding site that specifically binds to CD3 and a second antigen-binding site that specifically binds to an antigen different from CD3. It also provides the method for producing the bispecific antibody, and the use thereof.

### PRIORITY CLAIM

The present application is a U.S. National Phase application, filed under 35 U.S.C. § 371(c), of International Application No. PCT/CN2019/089032, filed May 29, 2019, which claims priority to, and the benefit of, PCT Application Number PCT/CN2018/088900, filed May 29, 2018, both of which are incorporated herein in their entirety by reference.

### INCORPORATION OF THE SEQUENCE LISTING

The contents of the text file submitted electronically herewith are incorporated herein by reference in their entirety: a computer readable format copy of the Sequence Listing (filename: CCPI_017_00US_SeqList_ST25.txt, date recorded Nov. 20, 2020, file size 36 kb).

### FIELD OF THE INVENTION

This application generally relates to antibodies. More specifically, the application relates to anti CD3/anti-CD20 bispecific antibody.

### BACKGROUND OF THE INVENTION

CD20 is an activated-glycosylated phosphoprotein expressed on the surface of B-lymphocytes. Antibody therapy with Rituximab, a chimeric anti-CD20 monoclonal antibody (also referred to as “mAb” hereinafter) approved by FDA in 1997, represents one of the most important progress in the treatment of lymphoproliferative disorders in the last 30 years. Particularly, in the combination with various chemotherapy/radiotherapy regimes, Rituximab has significantly improved all aspects of the survival statistics of B cell lymphoma and chronic lymphoid lymphoma (CLL) patients (Chu T W, Zhang R, Yang J, et al. A Two-Step Pretargeted Nanotherapy for CD20 Crosslinking May Achieve Superior Anti-Lymphoma Efficacy to Rituximab. Theranostics, 2015 Apr. 26; 5(8): 834-46).

During the past three decades, people made considerable progress in understanding of the protein structure and molecular function of CD20, therefore the new generation of anti-CD20 therapeutic antibodies have been generated and approved for clinical usage. Ofatumumab is a fully human anti-CD20 therapeutic antibody, which targets a different CD20 epitope of greater proximity to cell surface than Rituximab, resulting in a slower off-rate and more stable binding than Rituximab (Laurenti L, Innocenti I, Autore F, et al. New developments in the management of chronic lymphocytic leukemia: role of ofatumumab. Onco Targets Ther. 2016 Jan. 20: 9: 421-9). Nevertheless, the new generation of anti-CD20 monoclonal antibodies were not proven to be more significantly superior than Rituximab in efficacy and safety. For anti-CD20 mAb treatments, disease relapse or recurrence will still occur to all patients with follicular lymphoma and CLL, and about half of patients with aggressive B cell lymphoma, for example, diffuse large B cell lymphoma (Lim S H, Beers S A, French R R, et al. Anti-CD20 monoclonal antibodies: historical and future perspectives, Haematologica, 2010 January ;95(1):135-43). Thus, an unmet medical need is remained to develop new strategy of B cell-targeting therapeutics with distinct mechanism of action (MOA), such as bispecific antibody and chimeric antigen receptors (CARs)-T cell treatments.

A bispecific antibody targeting CD3 and a target antigen expressed on tumor cells could facilitate the killing of tumor by cytotoxic T cells. Such MOA approach was demonstrated to be successful by the approval of blinatumomab, an anti-CD3×CD19 bispecific antibody for the treatment of relapsed/refractory B cell acute lymphoblastic leukemia (ALL) (Sun L L, Ellerman D, Mathieu M. et al. Anti-CD20/CD3 T cell-dependent bispecific antibody for the treatment of B cell malignancies. Sci Transl Med. 2015 May 13;7(287):287ra70; D. Nagorsen, Kufer, P. A. Baeuerle, Bargou, Blinatumomab: A historical perspective. Pharmacol. Ther. 136, 334-342 (2012)), where the endogenous T cell killing of tumor cells was achievable without the need for ex vivo immune cell manipulation, providing advantages over cell-based therapies. Inspirited by this, we have generated novel anti CD3×CD20 bispecific antibody candidates for the treatment of CD20 expressing B cell malignancies, such as CLL and NHL.

Our anti-CD3×CD20 bispecific antibody was produced as humanized IgG4 in a knobs-into-holes format that avoids the formation of homodimers, in addition. IgG4 isotype minimized Fc mediated side effects. The bispecific antibody is cross-reactive to cynomolgus monkey CD3ε and CD20 antigens, allowing for appropriate preclinical testing. Further, the bispecific antibody demonstrates high potency and specificity in in vitro and in vivo B cell killing activity, and has manufacturing feasibility.

### SUMMARY OF THE INVENTION

These and other objectives are provided for by the present invention which, in a broad sense, is directed to compounds, methods, compositions and articles of manufacture that provide antibodies with improved efficacy. The benefits provided by the present invention are broadly applicable in the field of antibody therapeutics and diagnostics and may be used in conjunction with antibodies that react with a variety of targets. The present invention provides a bispecific antibody against CD3 and CD20. It also provides methods for generating the bispecific antibody and the use thereof, among others.

The following embodiments are contemplated and are non-limiting:

In some embodiments, the present disclosure provides a bispecific antibody or the antigen-binding portion thereof, comprising a first antigen-binding site that specifically binds to CD3 and a second antigen-binding site that specifically binds to an antigen different from CD3.

In some embodiments, the antigen different from CD3 is a tumor associated antigen.

In some embodiments, the tumor associated antigen comprise CD10, CD19, CD20, CD22, CD21, CD22, CD25, CD30, CD33, CD34, CD37, CD44v6, CD45, CD133, Fms-like tyrosine kinase 3 (FLT-3, CD135), chondroitin sulfate proteoglycan 4 (CSPG4, melanoma-associated chondroitin sulfate proteoglycan), epidermal growth factor receptor (EGER), Her2neu, Her3, IGFR, IL3R, fibroblast activating protein (PAP), CDCP1, Derlin1, Tenascin, frizzled 1-10, the vascular antigens VEGFR2 (KDR/FLK1), VEGER3 (FLT4, CD309), PDGER-alpha (CD140a), PDGFR-beta (CD 140b) Endoglin, CLEC14, Tem1-8, Tie2, A33, CAMPATH-1 (CDw52), Carcinoembryonic antigen (CEA), carboanhydrase IX (MN/CA IX), de2-7 EGFR, EGFRvIII, EpCAM, Ep-CAM, folatebinding protein, G250,Fms-like tyrosine kinase 3 (FLT-3, CD135), c-Kit (CD117), CSFIR (CD115), HLA-DR, IGFR, IL-2 receptor, IL3R, MCSP (Melanoma-associated cell surface chondroitin sulphate proteoglycane), Muc-1, Prostate-specific membrane antigen (PSMA), Prostate stem cell antigen (PSCA), Prostate specific antigen (PSA), and TAG-72.

In some embodiments, the tumor associated antigen is CD20.

In some embodiments, the first antigen-binding site specifically binds to CD3epsilon.

In some embodiments, the first antigen-binding site comprises in the heavy chain variable region a CDR (complementarity determining region) 1 of SEQ ID NO: 1, a CDR2 of SEQ IP3 NO: 2, and a CDR3 of SEQ ID NO: 3, and in the light chain variable domain a CDR1 of SEQ ID NO: 4, a CDR2 of SEQ ID NO: 5, and a CDR3 of SEQ ID NO: 6.

In some embodiments, the first antigen-binding site comprises in the heavy chain variable region:

(i) the amino acid sequence of SEQ ID NO: 13;

(ii) an amino acid sequence at least 85%, 90%, or 95% identical to the amino acid sequence of SEQ ID NO: 13; or

(iii) an amino acid sequence with addition, deletion ardor substitution of one or more amino acids compared with the amino acid sequence of SEQ ID NO: 13.

In some embodiments, the first antigen-binding site comprises in the light chain variable region:

(i) the amino acid sequence of SEQ ID NO: 14;

(ii) an amino acid sequence at least 85%, 90%, or 95% identical to the amino acid sequence of SEQ ID NO: 14; or

(iii) an amino acid sequence with addition, deletion and/or substitution of one or more amino acids compared with the amino acid sequence of SEQ ID NO: 14.

In some embodiments, the second antigen-binding site comprises in the heavy chain variable region a CDR1 of SEQ ID NO: 7, a CDR2 of SEQ ID NO: 8, and a CDR3 of SEQ ID NO: 9, and in the light chain variable domain a CDR1 of SEQ ID NO: 10, a CDR2 of SEQ ID NO: 11, and a CDR3 of SEQ ID NO: 12.

In some embodiments, the second antigen-binding site comprises in the heavy chain variable region:

(i) the amino acid sequence of SEQ ID NO: 15;

(ii) an amino acid sequence at least 85%, 90%, or 95% identical to the amino acid sequence of SEQ ID NO: 15; or

(iii) an amino acid sequence with addition, deletion and/or substitution of one or more amino acids compared with the amino acid sequence of SEQ ID NO: 15.

In some embodiments, the second antigen-binding site comprises in the light chain variable region:

(i) the amino acid sequence of SEQ ID NO: 16;

(ii) an amino acid sequence at least 85%, 90%, or 95% identical to the amino acid sequence of SEQ ID NO: 16; or

(iii) an amino acid sequence with addition, deletion and/or substitution of one or more amino acids compared with the amino acid sequence of SEQ ID NO: 16.

In some embodiments, the first antigen-binding site and the second antigen-binding site are fused by a linker.

In some embodiments, the bispecific antibody or the antigen-binding portion thereof further comprise a Fc region.

In some embodiments, the bispecific antibody or the antigen-binding portion thereof further comprise a human Fc region.

In some embodiments, the bispecific antibody or the antigen-binding portion thereof further comprise a human IgG Fc region.

In some embodiments, the bispecific antibody or the antigen-binding portion thereof further comprise a human IgG4 Fc region.

In some embodiments, the IgG4 Fc region is represented by SEQ ID NO: 42.

In some embodiments, the bispecific antibody or the antigen-binding portion thereof comprises a hinge sequence.

In some embodiments, the hinge sequence is represented by SEQ ID NO:41.

In some embodiments, the bispecific antibody or the antigen-binding portion thereof is in a knobs-into-holes format.

In some embodiments, the bispecific antibody or the antigen-binding portion thereof is a humanized antibody.

In some embodiments, the bispecific antibody or the antigen-binding portion thereof binds to cell surface human CD20 with a K_{D} of 1×10⁻⁷ M or less, as measured by FACS.

In some embodiments, the bispecific antibody of the antigen-binding portion thereof binds to cell surface, human CD3 with a K_{D} of 1×10⁻⁸ M or less, as measured by FACS.

In some embodiments, the bispecific antibody or the antigen-binding portion thereof induces T cell activation in the presence of target cells.

In some embodiments, the bispecific antibody or the antigen-binding portion thereof is effective in modulating killing of B-lymphocytes.

In some embodiments, the bispecific antibody or the antigen-binding portion thereof is stable, for instance, in DSF test, serum stability test and alkaline stress test.

In some embodiments, the bispecific antibody or the antigen-binding portion thereof is cross-reactive to cynomolgus monkey CD3 and CD20 antigens.

In some embodiments, the present disclosure provides an isolated nucleic acid molecule, comprising a nucleic acid sequence encoding the bispecific antibody as defined in the present disclosure.

In some embodiments, the present disclosure provides a vector comprising the isolated nucleic acid molecule as defined in the present disclosure.

In some embodiments, the present disclosure provides a host cell comprising the vector as defined in the present disclosure.

In some embodiments, the present disclosure provides a pharmaceutical composition comprising at least one antibody or antigen-binding portion thereof as defined in the present disclosure and a pharmaceutically acceptable carrier.

In some embodiments, the present disclosure provides a method for preparing a bispecific antibody or antigen-binding portion thereof as defined in the present disclosure, comprising the steps of:
expressing the bispecific antibody or antigen-binding portion thereof as defined in the present disclosure in the host cell of the present disclosure; and
isolating the bispecific antibody or antigen-binding portion thereof from the host cell.

In some embodiments, the present disclosure provides a method of modulating an immune response in a subject, comprising administering to the subject the bispecific antibody or antigen-binding portion thereof as defined in the present disclosure such that an immune response is modulated in the subject.

In some embodiments, T cell activation is induced in the presence of target cells.

In some embodiments, the present disclosure provides a method for treating abnormal cell growth in a subject, comprising, administering an effective amount of the antibody or antigen-binding portion thereof as defined in the present disclosure or the pharmaceutical composition of the present disclosure to the subject.

In some embodiments, the present disclosure provides a method for inhibiting growth of tumor cells in a subject, comprising administering an effective amount of the antibody or antigen-binding portion thereof as defined in the present disclosure or the pharmaceutical composition of the present disclosure to the subject.

In some embodiments, the cell is leukemic tumor cell.

In some embodiments, the present disclosure provides a method for reducing tumor cell metastasis in a subject, comprising administering an effective amount of the antibody or antigen-binding portion thereof as defined in the present disclosure or the pharmaceutical composition of the present disclosure to the subject.

In some embodiments, the present disclosure provides a method for treating or preventing diseases comprising proliferative disorders, autoimmune diseases, inflammatory disease or infectious diseases in a subject, comprising administering an effective amount of the antibody or antigen-binding portion thereof as defined in the present disclosure or the pharmaceutical. composition of the present disclosure to the subject.

In some embodiments, the proliferative disorders comprise cancer.

In some embodiments, the cancer comprises B-cell cancers.

In some embodiments, the cancer comprises leukemias and lymphomas.

In some embodiments, the cancer comprises chronic lymphoid lymphoma (CLL) and non-Hodgkin's lymphoma (NHL).

In some embodiments, the present disclosure provides use of the bispecific antibody or antigen-binding portion thereof as defined in the present disclosure in the manufacture of a medicament for modulating an immune response in a subject.

In some embodiments, the present disclosure provides use of the bispecific antibody or antigen-binding portion thereof as defined in the present disclosure in the manufacture of a medicament for treating abnormal cell growth in a subject.

In some embodiments, the present disclosure provides use of the bispecific antibody or antigen-binding portion thereof as defined in the present disclosure in the manufacture of a medicament for inhibiting growth of tumor cells in a subject.

In some embodiments, the present disclosure provides use of the bispecific antibody or antigen-binding portion thereof as defined in the present disclosure in the manufacture of a medicament for reducing tumor cell metastasis in a subject.

In some embodiments, the present disclosure provides use of the bispecific antibody or antigen-binding portion thereof as defined in the present disclosure in the manufacture of a medicament for treating or preventing proliferative disorders (such as cancers), autoimmune diseases, inflammatory disease or infectious diseases.

In some embodiments, the present disclosure provides use of the bispecific antibody or antigen-binding portion thereof as defined in the present disclosure in the manufacture of a diagnostic agent for diagnosing proliferative disorders (such as cancers), autoimmune diseases, inflammatory disease or infectious diseases.

In some embodiments, the bispecific antibody or antigen-binding portion thereof is useful for treating or preventing proliferative disorders (such as cancers), autoimmune diseases, inflammatory disease or infectious diseases.

In some embodiments, the bispecific antibody or antigen-binding portion thereof is useful for diagnosing proliferative disorders (such as cancers), autoimmune diseases, inflammatory disease or infectious diseases.

In some embodiments, the tumor or cancer comprises B-cell cancers.

In some embodiments, the tumor or cancer comprises leukemias and lymphomas.

In some embodiments, the tumor or cancer comprises chronic lymphoid lymphoma (CLL) and non-Hodgkin's lymphoma (NHL).

In some embodiments, the present disclosure provides a kit for treating or diagnosing proliferative disorders (such as cancers), autoimmune diseases, inflammatory disease or infections diseases, comprising a container comprising at least one antibody or antigen-binding portion thereof as defined in the present disclosure.

In some embodiments, the cancer comprises B-cell cancers.

The foregoing is a summary and thus contains, by necessity, simplifications, generalizations, and omissions of detail; consequently, those skilled in the art will appreciate that the summary is illustrative only and is not intended to be in any way limiting. Other aspects, features, and advantages of the methods, compositions and/or devices and/or other subject matter described herein will become apparent in the teachings set forth herein. The summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter. Further, the contents of all references, patents and published patent applications cited throughout this application are incorporated herein in entirety by reference.

### BRIEF DESCRIPTION OF THE FIGURES

FIG. 1A, Schematic diagram of T3U3-E4-1.uIgG4.SP.

FIG. 1B. SDS-PAGE of purified T3U3-E4-1.uIgG4.SP.

FIG. 1C. Analytic HPLC-SEC of purified T3U3-E4-1.uIgG4.SP.

FIG. 1D. MS analysis of purified T3U3-E4-1.uIgG4.SP.

FIG. 2A Binding of the bispecific antibodies to cell surface targets on Raji and Jurkat cells measured by FACS, respectively.

FIG. 2B. Binding, effects of the bispecific antibodies in simultaneous dual target binding determined by FACS. The simultaneous binding of bispecific antibody to both Raji and Jurket cells was evaluated by FACS, where the following Abs were added nub the 1:1 mixture of Raji and Jurkat cells. a). Mixture of parental anti-CD3 mAb and anti-CD20 mAb. b). BMK4. c). T3U3-E4-1.uIgG4.SP. The double positive events representing the bridged Raji and Jurkat cells by bispecific antibody were shown in the upper right part of FACS graph. d). The bar graph of % double positive events indicates that T3U3-E4-1.uIgG4.SP is more potent than BMK4 in the simultaneous dual target binding.

FIG. 3. Binding of T3U3-E4-1.uIgG4.SP to cell surface cyno-targets.

FIG. 4. Induction of T cell activation by the bispecific antibody in the presence of target cells. The activations of CD4+ and CD8+ T cells were measured by FACS for CD25 expression. The T cell activation mediated by bispecific antibody were strictly dependent on the presence of Raji cells (solid lines and symbols) and in a dose response mariner. In contrast, no T cell activation was observed in the absence of Raji cells (dotted lines and open symbols).

FIG. 5A. CD20 expression level on different B cell lines was detected with T3U3-E4-1.uIgG4.SP by FACS.

FIG. 5B. Cytotoxicity of three different B lymphoma cell lines (Ramos, Raji, and Namalwa) mediated by bispecific antibodies in two hour calcein release assay, measured by Envision.

FIG. 5C. Cytotoxicity of two different B lymphoma cell lines (Raji and Namalwa) mediated by bispecific antibodies in FACS based cytotoxicity assay.

FIG. 6A. DSF profiles of bispecific antibody (Left: T3U3-E4-1.uIgG4.SP; Right: BMK4).

FIG. 6B. Analytic HPLC SEC results show high purity and free of polymers and degradations of T3U3-E4-1.uIgG4.SP after incubation at 4° C. or 37° C. for 20 hours.

FIG. 7. Results of human serum stability test, measured by FACS binding to target cells.

FIG. 8. Results of alkaline stress test, measured by FACS binding to target cells.

FIG. 9A. Effects of bispecific antibodies on preventing Raji tumor growth in prophylactic tumor models.

FIG. 9B. Effects of bispecific antibodies on tumor inhibition in in vivo therapeutic tumor models. T3U3-E4-1.uIgG4.SP induced tumor growth inhibition at all tested doses, while BMK4 only at the highest dose. T3U3-E4-1.uIgG4.SP at 0.05 mg/kg was equally effective as Rituximab at 0.5 mg/kg and 5 mg/kg for significantly inhibiting tumor growth.

FIG. 9C. Effects of the bispecific antibody T3U3-E4-1.uIgG4.SP, Rituximab and BMK4 on inhibiting in vivo tumor growth and eradicating rumor at equal mole dose (=0.5 mg/kg).

### DETAILED DESCRIPTION OF THE INVENTION

While the present invention may be embodied in many different forms, disclosed herein are specific illustrative embodiments thereof that exemplify the principles of the invention. It should be emphasized that the present invention is not limited to the specific embodiments illustrated. Moreover, any section headings used herein are for organizational purposes only and are not to be construed as limiting the subject matter described.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. More specifically, as used in this specification and the appended claims, the singular forms “a,” “an” and “the” include plural referents unless the context clearly dictates otherwise. Thus, for example, reference to “a protein” includes a plurality of proteins; reference to “a cell” includes mixtures of cells, and the like. In this application, the use of “or” means “and/or” unless stated otherwise. Furthermore, the use of the term “comprising,” as well as other forms, such as “comprises” and “comprised,” is not limiting. In addition, ranges provided in the specification and appended claims include both end points and all points between the end points.

Generally, nomenclature used in connection with, and techniques of, cell and tissue culture, molecular biology, immunology, microbiology, genetics and protein and nucleic acid chemistry and hybridization described herein are those well-known and commonly used in the art. The methods and techniques of the present invention are generally performed according to conventional methods well known in the art and as described in various general and more specific references that are cited and discussed throughout the present specification unless otherwise indicated. See, e.g., Abbas et al., Cellular and Molecular Immunology, 6^{th} ed., W. B. Saunders Company (2010); Sambrook J. & Russell D. Molecular Cloning: A Laboratory Manual, 3rd ed., Cold Spring Harbor Laboratory Press, Cold Spring Harbor, N.Y. (2000); Ausubel et al., Short Protocols in Molecular Biology: A Compendium of Methods from Current Protocols in Molecular Biology, Wiley, John & Sons, Inc. (2002); Harlow and Lane Using Antibodies: A Laboratory Manual, Cold Spring Harbor Laboratory Press, Cold Spring Harbor, N.Y. (1998); and Coligan et al., Short Protocols in Protein Science, Wiley, John & Sons, Inc. (2003). The nomenclature used in connection with, and the laboratory procedures and techniques of, analytical chemistry, synthetic organic chemistry, and medicinal and pharmaceutical chemistry described herein are those well-known and commonly used in the art. Moreover, any section headings used herein are for organizational purposes only and are not to be construed as limiting the subject matter described.

### Definitions

In order to better understand the invention, the definitions and explanations of the relevant terms are provided as follows.

The term “antibody” or “Ab,” as used herein, generally refers to a Y-shaped tetrameric protein comprising two heavy (H) and two light (L) polypeptide chains held together by covalent disulfide bonds and non-covalent interactions. Light chains of an antibody may be classified into κ and λ light chain. Heavy chains may be classified into μ, 67 , γ, α and ε, which define isotypes of an antibody as IgM, IgD, IgG, IgA and IgE, respectively. In a light chain and a heavy chain, a variable region is lurked to a constant region via a “J” region of about 12 or more amino acids, and a heavy chain further comprises a “D” region of about 3 or more amino acids. Each heavy chain consists of a heavy chain variable region (V_{H}) and a heavy chain constant region (C_{H}). A heavy chain constant region consists of 3 domains (C_{H}1, C_{H}2 and C_{H}3). Each light chain consists of a light chain variable region (V_{L}) and a light chain constant region (C_{L}). V_{H} and V_{L} region can further be divided into hypervariable regions (called complementary determining regions (CDR)), which are interspaced by relatively conservative regions (called framework region (FR)). Each V_{H} and V_{L} consists of 3 CDRs and 4 FRs in the following order: FR1, CDR1, FR2, CDR2, FR3, CDR3, FR4 from N-terminal to C-terminal. The variable region (V_{H} and V_{L}) of each heavy/light chain pair forms antigen binding sites, respectively. Distribution of amino acids in various regions or domains follows the definition in Kabat Sequences of Proteins of Immunological Interest (National Institutes of Health, Bethesda, Md. (1987 and 1991)), or Chothia & Lesk (1987) J. Mol. Biol. 196:901-917; Choithia et al., (1989) Nature 342:878-883. Antibodies may be of different antibody isotypes, for example, IgG (e.g., IgG1, IgG2, IgG3 IgG4 subtype), IgA1, IgA2, IgD, IgE or IgM antibody.

The term “antigen-binding portion” or “antigen-binding fragment” of an antibody, which can be interchangeably used in the context of the application, refers to polypeptides comprising fragments of a full-length antibody, which retain the ability of specifically binding to an antigen that the full-length antibody specifically binds to, an /or compete with the full-length antibody for binding to the same antigen. Generally, see Fundamental Immunology, Ch. 7 (Paul, W., ed., the second edition, Raven Press, N.Y, (1989), which is incorporated herein by reference for all purposes. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarily determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding, fragment,” as used herein. In certain embodiments, an antigen-binding fragment of an antibody may contain at least one variable domain covalently linked to at least one constant domain. The variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule.

The term “CD3” as used herein, refers to the Cluster of Differentiation 3 protein derived from any vertebrate source, including mammals such as primates (e.g. humans, monkeys) and rodents (e.g., mice and rats). In mammals, the CD3 molecule is a multi-protein complex of six chains, including: a CD3gamma chain, a CD3delta chain, two CD3epsilon chains, and a homodimer of CD3zeta chains wherein the CD3zeta chain is the intracellular tail of CD3 molecule, and the CD3gamma, CD3delta and CD3epsilon chains all contain extracellular domain (ECD) expressed on surface of T cells. Exemplary sequence of human CD3 includes human CD3epsilon protein (NCBI Ref Seq No. NP_000724), human CD3 delta protein (NCBI Ref Seq No. NP_000723), and human CD3gamma protein (NCBI Ref Seq No. NP_000064). Exemplary sequence of human CD3 includes Macaca fascicularis (monkey) CD3epsilon protein (NCBI Ref Seq No, NP_001270544), Macaca fascicularis (monkey) CD 3delta protein (NCBI Ref Seq No. NP_001274617), Macaca fascicularis (monkey) CD3gamma protein (NCBI Ref Seq No, NP_001270839); mouse CD3epsilon protein (NCBI Ref Seq No. NP_031674), mouse CD3delta protein (NCBI Ref Seq No. NP_038515), mouse CD3gamma protein (NCBI Ref Seq No. AAA37400); Rattus norvegicus (Rat) CD3epsilon protein (NCBI RefSeq No. NP_001101610), Rattus norvegicus (Rat) CD3delta protein (NCBI Ref Seq No. NP_037301), Rattus norvegicus (Rat) CD3gamma protein (NCBI Ref Seq No. NP_001071114). In certain embodiments, CD3 used herein can also be recombinant CD3, for example, including recombinant CD3epsilon protein, recombinant CD3delta protein, and recombinant CD3gamma protein, which may optionally be expressed as a recombinant CD3 complex. The recombinant CD3 complex may be expressed on a cell surface, or alternatively may be expressed as a soluble form which is not associated on a cell surface.

The term “an antibody that binds CD3” or an “anti-CD3 antibody” as used herein includes antibodies and antigen-binding fragments thereof that specifically recognize a single CD3 subunit (e.g., epsilon, delta, gamma or zeta), as well as antibodies and antigen-binding; fragments thereof that specifically recognize as dimeric complex of two CD3 subunits (e.g., gamma/epsilon, delta/epsilon, and zeta/zeta CD3 dimers). The antibodies and antigen-binding fragments of the present invention may bind soluble CD3 and/or cell surface expressed CD3. Soluble CD3 includes natural CD3 proteins as well as recombinant CD3 protein variants such as, e.g., monomeric and dimeric CD3 constructs, that lack a transmembrane domain or are otherwise unassociated with a cell membrane.

As used herein, the term “cell surface-expressed CD3,” as used herein, refers to one or more CD3 protein(s) that is/are expressed on the surface of a cell in vitro or in vivo, such that at least a portion of a CD3 protein is exposed to the extracellular side of the cell membrane and is accessible to an antigen-binding portion of an antibody. “Cell surface-expressed CD3” includes CD3 proteins contained within the context of a functional T cell receptor in the membrane of a cell. The expression “cell surface-expressed CD3” includes CD3 protein expressed as part of a homodimer or heterodimer on the surface of a cell (e.g., gamma/epsilon, delta/epsilon, and zeta/zeta CD3 dimers). The expression, “cell surface-expressed CD3” also includes aa. CD3 chain (e.g., CD3-epsilon, CD3-delta or CD3-gamma) that is expressed by itself, without other CD3 chain types, on the surface of a cell. A “cell surface-expressed CD3” can comprise or consist of a CD3 protein expressed on the surface of a cell which normally expresses CD3 protein, Alternatively, “cell surface-expressed CD3” can comprise or consist of CD3 protein expressed on the surface of a cell that normally does not express human CD3 on its surface but has been artificially engineered to express CD3 on its surface. As used herein, the expression “anti-CD3 antibody” includes both monovalent antibodies with a single specificity, as well as bispecific antibodies comprising a first antigen-binding site that binds CD3 and a second antigen-binding site that binds a second (target) antigen, wherein the anti-CD3 antigen-binding site comprises any of the HCVR/LCVR or CDR sequences as set forth in Table 1 or Table 2 herein. Examples of anti-CD3 bispecific antibodies are described elsewhere herein. The term “antigen-binding molecule” includes antibodies and antigen-binding fragments of antibodies, including, bispecific antibodies. Exemplary anti-CD3 antibodies are also described in US 2007/0280945A1; and in PCT international Application No. PCT/US13/60511, filed on Sep. 19, 2013, which is herein incorporated by reference in its entirety.

The term “CD3epsilon” or “CD3ε” as used herein is intended to encompass any form of CD3epsilon, for example, 1) native unprocessed CD3epsilon molecule, “full-length” CD3epsilon chain or naturally occurring variants of CD3epsilon, including, for example, splice variants or allelic variants; 2) any form of CD3epsilon that results from processing in the cell; or 3) full length, a fragment (e.g., a truncated form, an extracellular/transmembrane domain) or a modified form (e.g. a mutated form, a glycosylated/PEGylated, a Histag/immunofluorescence fused form) of CD3epsilon subunit generated through recombinant method.

The term “anti-CD3epsilon antibody” refers to an antibody that is capable of specific binding to CD3epsilon.

The term “CD20,” as used herein, refers to an activated-glycosylated phosphoprotein expressed on the surface of B-lymphocytes. The human CD20 protein has the amino acid sequence as in GenBank Accession No. NP_690605.1.

The term “anti-CD20 antibody,” as used herein, refers to an antibody that specifically binds to CD20. An “anti-CD20 antibody” may include monovalent antibodies with a single specificity, such as Rituxan (rituximab), and bispecific antibody. Exemplary anti-CD20 antibodies are described in U.S. Pat. No. 7,879,984B2 and PCT International Application No. PCT/US13/60511, each incorporated by reference herein.

The term “bivalent,” as used herein refers to an antibody or an antigen-binding fragment having two antigen-binding sites the term “monovalent” refers to an antibody or an antigen-binding fragment having only one single antigen-binding site; and the term “multivalent” refers to an antibody or an antigen-binding fragment having multiple antigen-binding sites. In some embodiments, the antibody or antigen-binding fragment thereof is bivalent.

As used herein, a “bispecific” antibody refers to an artificial antibody which has fragments derived from two different monoclonal antibodies and is capable of binding to two different epitopes. The two epitopes may present on the same antigen, or they may present on two different antigens.

The term “bispecific antigen-binding molecule” means a protein, polypeptide or molecular complex comprising at least a first antigen-binding domain (also referred to as a first antigen-binding site herein) and a second antigen-binding domain (also referred to as a second antigen-binding site herein). In some embodiment, the “bispecific antigen-binding molecule” is a “bispecific antibody.” Each antigen-binding domain within the bispecific antibody comprises at least one CDR that alone, or in combination with one or more additional CDRs and/or FRs, specifically binds to a particular antigen. In the context of the present invention, the first antigen-binding site specifically binds to a first antigen (e.g., CD3), and the second antigen-binding, site specifically binds to a second, distinct antigen (e g., CD20).

The term “anti-CD3/anti-CD20 antibody,” “anti-CD3/anti-CD20 bispecific antibody,” “antibody against CD3 and CD20,” “anti-CD3×CD20 bispecific antibody,” “CD3×CD20 antibody,” as used herein interchangeably, refers to a bispecific antibody that specifically binds to CD3 and CD20.

The term “monoclonal antibody” or “mAb”, as used herein, refer to a preparation of antibody molecules of single molecular composition. A monoclonal antibody displays a single binding specificity and affinity for a particular epitope.

The term “human antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as, a mouse, have been grafted onto human framework sequences.

The term “humanized antibody” is intended, to refer to antibodies in which CDR sequences derived from the germline of another mammalian species, such as a mouse, have been grafted onto human framework sequences. Additional framework region modifications may be made within the human framework sequences.

The term “chimeric antibody,” as used herein, refers to an antibody in which the variable region sequences are derived from one species and the constant region sequences are derived from another species, such as an antibody in which the variable region sequences are derived from a mouse antibody and the constant region sequences are derived from a human antibody.

The term “recombinant antibody,” as used herein, refers to an antibody that is prepared, expressed, created or isolated by recombinant means, such as antibodies isolated from an animal that is transgenic for another species' immunoglobulin genes, antibodies expressed using a recombinant expression vector transfected into a host cell, antibodies isolated from a recombinant, combinatorial antibody library, or antibodies prepared, expressed, created or isolated by any other means that involves splicing of immunoglobulin gene sequences to other DNA sequences.

The term “Ka,” as used herein, is intended to refer to the association rate of a particular antibody-antigen interaction, whereas the term “Kd” as used herein, is intended to refer to the dissociation rate of a particular antibody-antigen interaction. Kd values for antibodies can be determined using methods well established in the art. The term “K_{D}” as used herein, is intended to refer to the dissociation constant of a particular antibody-antigen interaction, which is obtained from the ratio of Kd to Ka (i.e., Kd/Ka) and is expressed as a molar concentration (M). A preferred method for determining the Kd of an antibody is by using surface plasmon resonance, preferably using a biosensor system such as a Biacore® system.

The term “high affinity” for an IgG antibody, as used herein, refers to an antibody having a K_{D} of 1×10⁻⁷ M or less, more preferably 5×10⁻⁸ M or less, even more preferably 1×10⁻⁸ M or less, even more preferably 5×10⁻⁹ M or less and even more preferably 1×10⁻⁹ M or less for a target antigen.

The term “EC₅₀,” as used herein, which is also termed as “half maximal effective concentration” refers to the concentration of a drug, antibody or toxicant which induces a response halfway between the baseline and maximum after a specified exposure time. In the context of the application, EC₅₀ is expressed in the unit of “nM”.

The term “compete for binding,” as used herein, refers to the interaction of two antibodies in their binding to a binding target. A first antibody competes for binding with a second antibody if binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not, be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope, whether to the same, greater, or lesser extent, the antibodies are said to “cross-compete” with each other for binding of their respective epitope(s).

The ability of “inhibit binding,” as used herein, refers to the ability of an antibody or antigen-binding fragment thereof to inhibit the binding of two molecules leg, human CD3/CD20 and human anti-CD3/anti-CD20 antibody) to any detectable level. In certain embodiments, the binding of the two molecules can be inhibited at least 50% by the antibody or antigen-binding fragment thereof. In certain embodiments, such, an inhibitory effect may be greater than 60%, greater than 70%, greater than 80%, or greater than 90%.

The term “epitope,” as used herein, refers to a portion on antigen that an immunoglobulin or antibody specifically binds to. “Epitope” is also known as “antigenic determinant”. Epitope or antigenic determinant generally consists of chemically active surface groups of a molecule such as amino acids, carbohydrates or sugar side chains, and generally has a specific three-dimensional structure and a specific charge characteristic. For example, an epitope generally comprises at least 3, 4, 5, 6, 7, 8, 9, 10, 11, 12, 13, 14 or 15 consecutive or non-consecutive amino acids in a unique steric conformation, which may be “linear” or “conformational”. See, for example, Epitope Mapping Protocols in Methods in Molecular Biology, Vol. 66, a G. E. Morris, Ed. (1996). In a linear epitope, all the interaction sites between a protein and an interaction molecule (e.g., an antibody) are present linearly along the primary amino acid sequence of the protein. In a conformational epitope, the interaction sites span over amino acid residues that are separate from each other in a protein. Antibodies may be screened depending on competitiveness of binding to the same epitope by conventional techniques known by a person skilled in the art. For example, study on competition or cross-competition may be conducted to obtain antibodies that compete or cross-compete with each other for binding to antigens (e.g. RSV fusion protein). High-throughput methods for obtaining antibodies binding to the same epitope, which are based on their cross-competition, are described in an international patent application WO 03/48731.

The term “isolated,” as used herein, refers to a state obtained from natural state by artificial means. If a certain “isolated” substance or component is present in nature, it is possible because its natural environment changes, or the substance is isolated from natural environment, or both. For example, a certain un-isolated polynucleotide or polypeptide naturally exists in a certain living animal body, and the same polynucleotide or polypeptide with a high purity isolated from such a natural state is called isolated polynucleotide or polypeptide. The term “isolated” excludes neither the mixed artificial or synthesized substance nor other impure substances that do not affect the activity of the isolated substance.

The term “isolated antibody,” as used herein, is intended to refer to an antibody that is substantially free of other antibodies having different antigenic specificities (e.g., an isolated antibody that specifically binds an CD3/CD20 protein is substantially free of antibodies that specifically bind antigens other than CD3/CD20 proteins). An isolated antibody that specifically binds a human CD3/CD20 protein may, however, have cross- reactivity to other antigens, such as CD3/CD20 proteins from other species. Moreover, an isolated antibody can be substantially free of other cellular material and/or chemicals.

The term “vector,” as used herein, refers to a nucleic acid vehicle which can have a polynucleotide inserted therein. When the vector allows for the expression of the protein encoded by the polynucleotide inserted therein, the vector is called an expression vector. The vector can have the carried genetic material elements expressed in a host cell by transformation, transduction, or transfection into the host cell. Vectors are well known by a person skilled in the art, including, but not limited to plasmids, phages, cosmids, artificial chromosome such as yeast artificial chromosome (YAC), bacterial artificial chromosome (BAC) or Pl-derived artificial chromosome (PAC); phage such as λ phage or M13 phage and animal virus. The animal viruses that can be used as vectors, include, but are not limited to, retrovirus (including lentivirus), adenovirus, adeno-associated virus, herpes virus (such as herpes simplex virus), pox virus, baculovirus, papillornavirus, papova virus (such as SV40). A vector may comprise multiple elements for controlling expression, including, but not limited to, a promoter sequence, a transcription initiation sequence, an enhancer sequence, a selection element and a reporter gene. In addition, a vector may comprise origin of replication.

The term “host cell,” as used herein, refers to a cellular system which can be engineered to generate proteins, protein fragments, or peptides of interest. Host cells include, without limitation, cultured cells, e.g., mammalian cultured cells derived from rodents (rats, mice, guinea pigs, or hamsters) such as CHO, BHK, NSO, SP2/0, YB2/0; or human tissues or hybridoma cells, yeast cells, and insect cells, and cells comprised within a transgenic animal or cultured tissue. The term encompasses not only the particular subject cell but also the progeny of such a cell. Because certain modifications may occur in succeeding generations due to either mutation or environmental influences, such progeny may not be identical to the parent cell, but are still included within the scope of the term “host cell.”

The term “identity,” as used herein, refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) are preferably addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York; Oxford University Press; Biocomputing Informatics and Genome Projects, (Smith, D. W., ed.), 1993, New York; Academic Press; Computer Analysis of Sequence Data, Part I, (Griffin, A, M., and Griffin, H. G., eds.), 1994, New Jersey; Humana Press; von Heinje, G., 1987, Sequence Analysis in Molecular Biology, New York; Academic Press; Sequence Analysis Primer, (Gribskov, M. and Devereux, eds.)., 1991, New York; M. Stockton Press; and Carillo et al, 1988, SIAMJ. Applied Math. 48:1073.

The term “immunogenicity,” as used herein, refers to ability of stimulating the formation of specific antibodies or sensitized lymphocytes in organisms. It not only refers to the property of an antigen to stimulate a specific immunocyte to activate, proliferate and differentiate so as to finally generate immunologic effector substance such as antibody and sensitized lymphocyte, but also refers to the specific immune response that antibody or sensitized T lymphocyte can be formed in immune system of an organism after stimulating the organism with an antigen. Immunogenicity is the most important property of an antigen. Whether an antigen can successfully induce the generation of an immune response in a host depends on three factors, properties of an antigen, reactivity of a host, and immunization means.

The term “transfection,” used herein, refers to the process by which nucleic acids are introduced into eukaryotic cells, particularly mammalian cells. Protocols and techniques for transfection include but not limited to lipid transfection and chemical and physical methods such as electroporation. A number of transfection techniques are well known in the art and are disclosed herein. See, e.g., Graham et al., 1973, Virology 52:456; Sambrook et al., 2001, Molecular Cloning: A Laboratory Manual, supra; Davis et al., 1986, Basic Methods in Molecular Biology, Elsevier; Chu et al, 1981, Gene 13:197. In a specific embodiment of the invention, human CD3/CD20 gene was transfected into 293F cells.

The term “hybridoma” and the term “hybridoma cell line,” as used herein, may be used interchangeably. When the term “hybridoma” and the term “hybridoma cell line” are mentioned, they also include subclone and progeny cell of hybridoma.

The term “SPR” or “surface plasmon resonance,” as used herein, refers to and includes an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Example 5 and Jönsson, U., et al. (1993) Ann. Biol. Clin. 51:19-26; Jönsson, U., et al. (1991) Biotechniques 11:620-627, Johnsson, B., et al. (1995) J. Mol. Recognit. 8:125-131; and Jobnnson, B., et al. (1991) Anal. Biochem, 198:268-277.

The term “fluorescence-activated cell sorting” or “FACS,” as used herein, refers to a specialized type of flow cytometry. It provides a method for sorting a heterogeneous mixture of biological cells into two or more containers, one cell at a time, based upon the specific light scattering and fluorescent characteristics of each cell (FlowMetric. “Sorting Out Fluorescence Activated Cell Sorting”. Retrieved 2017-11-09.). Instruments for carrying out FACS are known to those of skill in the art and are commercially available to the public. Examples of such instruments include FACS Star Plus, FACScan and FACSort instruments from Becton Dickinson (Foster City, Calif.) Epics C from Coulter Epics Division (Hialeah, Fla.) and MoFlo from Cytomation (Colorado Springs, Colo.).

The term “antibody-dependent cell-mediated cytotoxicity” or “ADCC,” as used herein, refers to a form of cytotoxicity in which secreted Ig bound onto Fc receptors (FcRs) present on certain cytotoxic cells (e.g. Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev Immunol 9:457-92 (1991). To assess ADCC activity of a molecule of interest, an in vitro ADCC assay, such as that described in U.S. Pat. Nos. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. PNAS (USA) 95:652-656 (1998).

The term “complement dependent cytotoxicity” or “CDC” refers to the lysis of a target cell in the presence of complement. Activation of the classical complement pathway is initiated by the binding of the first component of the complement system (Clq) to antibodies (of the appropriate subclass) which are bound to their cognate antigen. To assess complement activation, a CDC assay, e.g. as described in Gazzano-Santoro et al., J. Immunol, Methods 202, 163 (1996), may be performed.

The term “subject” includes any human or nonhuman animal, preferably humans.

The term “cancer,” as used herein, refers to any or a tumor or a malignant cell growth, proliferation or metastasis-mediated, solid tumors and non-solid tumors such as leukemia and initiate a medical condition.

The term “treatment,” “treating” or “treated,” as used herein in the context of treating a condition, pertains generally to treatment and therapy, whether of a human or an animal, in which some desired therapeutic effect is achieved, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included. For cancer, “treating” may refer to dampen or slow the tumor or malignant cell growth, proliferation, or metastasis, or some combination thereof. For tumors, “treatment” includes removal of all or part of the tumor, inhibiting or slowing tumor growth and metastasis, preventing or delaying the development of a tumor, or some combination thereof.

The term “an effective amount,” as used herein, pertains to that amount of an active compound, or a material, composition or dosage from comprising an active compound, which is effective for producing some desired therapeutic effect, commensurate with a reasonable benefit/risk ratio, when administered in accordance with a desired treatment regimen. For instance, the “an effective amount,” when used in connection with treatment of CO3/CD20-related diseases or conditions, refers to an antibody or antigen-binding portion thereof in an amount or concentration effective to treat the said diseases or conditions.

The term “prevent,” “prevention” or “preventing,” as used herein, with reference to a certain disease condition in a mammal, refers to preventing or delaying the onset of the disease, or preventing the manifestation of clinical or subclinical symptoms thereof.

The term “pharmaceutically acceptable,” as used herein, means that the vehicle, diluent, excipient and/or salts thereof, are chemically and/or physically is compatible with other ingredients in the formulation, and the physiologically compatible with the recipient.

As used herein, the term “a pharmaceutically acceptable carrier and/or excipient” refers to a carrier and/or excipient pharmacologically and/or physiologically compatible with a subject and an active agent, which is well known in the art (see, e.g., Remington's Pharmaceutical Sciences. Edited by Gennaro A R, 19th ed. Pennsylvania: Mack Publishing Company, 1995), and includes, but is not limited to pH adjuster, surfactant, adjuvant and ionic strength enhancer. For example, the pH adjuster includes, but is not limited to, phosphate buffer; the surfactant includes, but is not limited to, cationic, anionic, or non-ionic surfactant, e.g., Tween-80; the ionic strength enhancer includes, but is not limited to, sodium chloride.

As used herein, the term “adjuvant” refers to a non-specific immunopotentiator, which can enhance immune response to an antigen or change the type of immune response in an organism when it is delivered together with the antigen to the organism or is delivered to the organism in advance. There are a variety of adjuvants, including, but not limited to, aluminium adjuvants (for example, aluminum hydroxide), Freund's adjuvants (for example, Freund's complete adjuvant and Freund's incomplete adjuvant), coryne bacterium parvum, lipopolysaccharide, cytokines, and the like. Freund's adjuvant is the most commonly used adjuvant in animal experiments now. Aluminum hydroxide adjuvant is more commonly used in clinical trials.

### Bispecific Antibodies and Antigen-Binding Fragments Thereof

In certain embodiments, the antibodies and antigen-binding fragments thereof provided herein are bispecific. In some embodiments, the bispecific antibodies and antigen-binding fragments thereof provided herein has a first specificity for CD3, for example CD3epsilon, and a second specificity different from CD3. In some embodiments, the second specificity is for a second antigen different from CD3epsilon and whose presence in proximity to CD3epsilon-expressing T cells is desirable for the second antigen to be recognized by immune system. For example, bringing CD3epsilon-expressing T cells in close proximity to a tumor antigen or a pathogen antigen and hence promoting recognition or elimination of such an antigen by the immune system.

In certain embodiments, the second specificity is for a tumor associated antigen or an epitope thereof. The term “tumor associated antigen” refers to a target antigen expressed by tumor cells, however may be expressed by the cognate cell (or healthy cells) prior to transforming into a tumor. In some embodiments, the tumor associated antigens can be presented only by tumor cells and not by normal, i.e. non-tumor cells. In some other embodiments, the tumor associated antigens can be exclusively expressed on tumor cells or may represent a tumor specific mutation compared to non-tumor cells. In some other embodiments, the tumor associated antigens can be found in both tumor cells and non-tumor cells, but is overexpressed on tumor cells when compared to non-tumor cells or are accessible for antibody binding in tumor cells due to the less compact structure of the tumor tissue compared to non-tumor tissue. In some embodiments, the tumor associated antigen is located on the vasculature of a tumor.

Illustrative examples of a tumor associated antigen are CD10, CD19, CD20, CD22, CD21, CD22, CD25, CD30, CD33, CD34, CD37, CD44v6, CD45, CD133, Fms-like tyrosine kinase 3 (FLT-3, CD135), chondroitin sulfate proteoglycan 4 (CSPG4, melanoma-associated chondroitin sulfate proteoglycan). Epidermal growth factor receptor (EGFR), Her2neu, Her3, IGFR, , IL3R, fibroblast activating protein (FAP), CDCP1, Derlin1, Tenascin, frizzled 1-10, the vascular antigens VEGFR2 (KDR/FLK1), VEGFR3 (FLT4, CD309), PDGFR-alpha (CD140a), PDGFR-beta (CD140b) Endoglin, CLEC14, Tem1-8, and Tie2. Further examples may include A33, CAMPATH-1 (CDw52), Carcinoembryonic antigen (CEA), Carboanhydrase IX (MN/CA IX), de2-7 EGFR, EGFRvIII, EpCAM, Ep-CAM, Folate-binding protein, G250,Fms-like tyrosine kinase 3 (FLT-3, CD135), c-Kit (CD117), CSF1R (CD115), HLA-DR, IGFR, IL-2 receptor, IL3R, MCSP (Melanoma-associated cell surface chondroitin sulphate proteoglycane), Muc-1, Prostate-specific membrane antigen (PSMA), Prostate stem cell antigen (PSCA), Prostate specific antigen (PSA), and TAG-72.

In certain embodiments, the second specificity is for an infectious disease-associated antigen or an epitope thereof. Non-limiting examples of infectious disease-associated antigens include, e.g., an antigen that is expressed on the surface of a virus particle, or preferentially expressed on a cell that is infected with a virus, wherein the virus is selected from the group consisting of HIV, hepatitis (A, B or C), herpes virus (e.g., HSV-1, HSV-2, CMV, HAV-6, VZV, Epstein Barr virus), adenovirus, influenza virus, flavivirus, echovirus, rhinovirus, coxsackie virus, coronavirus, respiratory syncytial virus, mumps virus, rotavirus, measles virus, rubella virus, parvovints, vaccinia virus, HTLV, dengue virus, papillomavirus- molluscum virus, poliovirus, rabies virus, JC virus, and arboviral encephalitis virus. Alternatively, the target antigen can be an antigen that is expressed on the surface of a bacterium, or preferentially expressed on a cell that is infected with a bacterium, wherein the bacterium is selected from the group consisting of chlamydia, rickettsia, mycobacteria, staphylococci, streptococci, pneumonococci, meningococci, gonococci, klebsiella, proteuli, serratia, pseudomonas, legionella, diphtheria, salmonella, bacilli, cholera, tetanus, botulism, anthrax, plague, leptospira, and Lyme disease bacteria. In certain embodiments, the target antigen is an antigen that is expressed on the surface, of a fungus, or preferentially expressed on a cell that is infected with a fungus, wherein the fungus is selected from the group consisting of Candida (albicans, krusei, glabrata, tropicalis, etc.), Crytococcus neoformans, Aspergillus (fumigants, niger, etc.), Mucorales (mucor, absidia, rhizopus, etc.), Sporothrix schenkii, Blastomyces dermatitidis, Paracoccidioides brasiliensis, Coccidioides immitis, and Histoplasma capsulatum. In certain embodiments, the target antigen is an antigen that is expressed on the surface of a parasite, or preferentially expressed on a cell that is infected with a parasite, wherein the parasite is selected from the group consisting of Entamoeba histolytica, Balantidium coli, Naegleriafowleri, Acanthamoeba sp., Giardia lambia, Cryptosporidium sp., Pneuntocystis carinii, Plasmodium vivax, Babesia microti, Trypanosoma bucei, Trypanosoma cruzi, Leishmania donovani, Toxoplasma gondii, Nippostrongylus brasiliensis, Taenia crassiceps, and Brugia malayi. Non-limiting examples of specific pathogen-associated antigens include, e.g., HIV gp120, HIV CD4. hepatitis B glucoprotein L, hepatitis B glucoprotein M, hepatitis B glucoprotein S, hepatitis C E1, hepatitis C E2, hepatocyte-specific protein, herpes simplex virus gB, cytomegalovirus gB, and HTLV envelope protein.

According to certain exemplary embodiments, the present invention includes a bispecific antibody or the antigen-binding portion thereof, comprising a first antigen-binding site that specifically binds to CD3 and a second antigen-binding site that specifically binds to CD20. Such antibodies may be referred to herein as, e.g., “anti-CD3/anti-CD20,” or “anti-CD3/CD20,” or “anti-CD3×CD20” or “CD3×CD20” bispecific antibodies, or other similar terminology.

The bispecific antibody of the invention binds to human CD3 with high affinity. The binding of an antibody of the invention to CD3 can be assessed using one or more techniques well established in the an, for instance, ELISA. The binding specificity of an antibody of the invention can also be determined by monitoring binding of the antibody to cells expressing a CD3 protein, e.g., flow cytometry. For example, an antibody can be tested by a flow cytometry assay in which the antibody is reacted with a cell line that expresses human CD3, such as CHO cells that have been transfected to express CD3 on their cell surface. Additionally or alternatively, the binding of the antibody, including the binding kinetics (e.g., K_{D} value) can be tested in BIAcore binding assays. Still other suitable binding assays, include ELISA or FACS assays, for example using a recombinant CD3 protein, For instance, an antibody of the invention binds to a human CD3 protein with a K_{D} of 5×10⁻⁷ M or less, binds to a human CD3 protein with a K_{D} of 2×10⁻⁷ M or less, binds to a human CD3 protein with a K_{D} of 1×10⁻⁷ M or less, binds to a human CD3 protein with a K_{D} of 5×10⁻⁸ M or less, binds to a human CD3 protein with a of 2×10⁻⁸ M or less, binds to a human CD3 protein with a K_{D} of 1×10⁻⁸ M or less, binds to a human CD3 protein with a K_{D} of 5×10⁻⁹ M or less, binds to a human CD3 protein with a K_{D} of 4×10⁻⁹ M or less, binds to a human CD3 protein with a K_{D} of 3×10⁻⁹ M or less, binds to a human CD3 protein with a K_{D} of 2×10⁻⁻⁹ M or less, binds to a human CD3 protein with a K_{D} of 1×10⁻⁹ M or less, binds to a human CD3 protein with a K_{D} of 5 ×10⁻¹⁰ M or less, or binds to a human CD3 protein with a K_{D} of 1×10⁻¹⁰ M or less, as measured by FACS.

The bispecific antibody of the invention binds to human CD20 with high affinity. The binding of an antibody of the invention to CD20 can be assessed using one or more techniques well established in the art, for instance, ELISA. The binding specificity of an antibody of the invention can also be determined by monitoring binding of the antibody to cells expressing a CD20 protein, e.g., flow cytometry. For example, an antibody can be tested by a flow cytometry assay in which the antibody is reacted with a cell line that expresses human CD20, such as CHO cells that have been transfected to express CD20 on their cell surface. Additionally or alternatively, the binding of the antibody, including the binding kinetics (e.g., K_{D} value) can be tested in BIAcore binding assays. Still other suitable binding assays include ELISA or FACS assays, for example using a recombinant CD20 protein. For instance, an antibody of the invention binds to a human CD20 protein with a K_{D} of 5×10⁻⁷ or less, binds, to a human CD20 protein with a K_{D} of 2×10⁻⁷ M or less, binds to a human CD20 protein with a K_{D} of 1×10⁻⁷ M or less, binds to a human CD20 protein with a K_{D} of 5×10⁻⁸ M or less, binds to a human. CD20 protein with a K_{D} of 2×10⁻⁸ M or less, binds to a human CD20 protein with a K_{D} of 1×10⁻⁸ M or less, binds to a human CD20 protein with a K_{D} of 5×10⁻⁹ M or less, binds to a human CD20 protein with a K_{D} of 4×10⁻⁹ M or less, binds to a human CD20 protein with a K_{D} of 3×10⁻⁹ M or less, binds to a human CD20 protein with a K_{D} of 2×10⁻⁹ M or less, binds to a human CD20 protein with a K_{D} of 1×10⁻⁹ M or less, binds to a human CD20 protein with a K_{D} of 5×10⁻¹⁰ M or less, or binds to a human CD20 protein with a K_{D} of 1×10⁻¹⁰ M or less, as measured by FACS.

### The First Antigen-Binding Site That Specifically Binds to CD3

In one embodiment, the first antigen-binding site comprises in the heavy chain variable region a CDR (complementarity determining region) 1 of SEQ ID NO: 1, a CDR2 of SEQ ID NO: 2, and a CDR3 of SEQ ID NO: 3, and in the light chain variable domain a CDR1 of SEQ ID NO: 4, a CDR2 of SEQ ID NO: 5, and a CDR3 of SEQ ID NO: 6.

In one embodiment, the first antigen-binding site comprises in the heavy chain variable region comprises:

(i) the amino acid sequence of SEQ ID NO: 13;

(ii) an amino acid sequence at least 85%, 90%, or 95% identical to the amino acid sequence of SEQ ID NO: 13; or

(iii) an amino acid sequence with addition, deletion and/or substitution of one or more amino acids compared with the amino acid sequence of SEQ ID NO: 13.

In one embodiment, the first antigen-binding site comprises in the light chain variable region comprises:

(i) the amino acid sequence of SEQ ID NO: 14;

(ii) an amino acid sequence at least 85%, 90%, or 95% identical to the amino acid sequence of SEQ ID NO: 14; or

(iii) an amino acid sequence with addition, deletion and/or substitution of one or more amino acids compared with the amino acid sequence of SEQ ID NO: 14,

### The second Antigen-Binding Site That Specifically Binds to CD20

In one embodiment, the second antigen-binding site comprises in the heavy chain variable region a CDR1 of SEQ ID NO: 7, a CDR2 of SEQ ID NO: 8, and a CDR3 of SEQ ID NO: 9, and in the light chain variable domain a CDR1 of SEQ ID NO: 10, a CDR2 of SEQ ID NO: 11, and a CDR3 of SEQ ID NO: 12.

In one embodiment, the second antigen-binding site comprises in the heavy chain variable region comprises:

(i) the amino acid sequence of SEQ ID NO: 15;

(ii) an amino acid sequence at least 85%, 90%, or 95% identical to the amino acid sequence of SEQ ID NO: 15; or

(iii) an amino acid sequence with addition, deletion and/or substitution of one or more amino acids compared with the amino acid sequence of SEQ ID NO: 15:

In one embodiment, the second antigen-binding site comprises in the light chain variable region comprises:

(i) the amino acid sequence of SEQ NO: 16;

(ii) an amino acid sequence at least 85%, 90%, or 95% identical to the amino acid sequence of SEQ ID NO: 16; or

(iii) an amino acid sequence with addition, deletion and/or substitution of one or more amino acids compared with the amino acid sequence of SEQ ID NO: 16.

The assignment of amino acids to each CDR may be in accordance with one of the numbering schemes provided by Kabat et al. (1991) Sequences of Proteins of Immunological Interest (5^{th} Ed.), US Dept. of Health and Human Services, PHS, NIH, NTH Publication no. 91-3242; Chothia et al., 1987, PMID: 3681981: Chothia et al., 1989, PMID: 2687698; MacCallum et al., 1996, PMID: 8876650; or Dubel, Ed. (2007) Handbook of Therapeutic Antibodies, 3^{rd} Ed., Wily-VCH Verlag GmbH and Co. unless otherwise noted.

Variable regions and CDRs in an antibody sequence can be identified according to general rules that have been developed in the art (as set out above, such as, for example, the Kabat numbering system) or by aligning the sequences against a database of known variable regions. Methods for identifying these regions are described in Kontermann and Dubel, eds., Antibody Engineering, Springer, New York, N.Y., 2001 and Dinarello et al., Current Protocols in Immunology, John Wiley and Sons Inc., Hoboken, N.J. 2000. Exemplary databases of antibody sequences are described in, and can be accessed through, the “Abysis” website at www.bioinf.org.uk/abs (maintained by A. C. Martin in the Department of Biochemistry & Molecular Biology University College London, London, England) and the VBASE2 website at www.vbase2.org, as described in Retter et al., Nucl. Acids Res., 33 (Database issue): D671 -D674 (2005). Preferably sequences are analyzed using the Abysis database, which is sequence data from Kabat, IMGT and the Protein Data Bank (PDB) with structural data from the PDB. See Dr. Andrew C. R. Martin's book chapter Protein Sequence and Structure Analysis of Antibody Variable Domains. In: Antibody Engineering Lab Manual (Ed.: Duebel, S. and Kontermann, R., Springer-Verlag, Heidelberg, ISBN-13: 978-3540413547, also available on the website bioinforg.uk/abs). The Abysis database website further includes general rules that have been developed for identifying CDRs which can be used in accordance with the teachings herein. Unless otherwise indicated, all CDRs set forth herein are derived according to the Abysis database website as per Kabat.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percentage of identity between two amino acid sequences can be determined by the algorithm of Needleman and Wunsch (J. Mol. Biol. 48:444-453 (1970)) which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the antibody molecules of the invention. To obtain gapped alignments for comparison purposes, Gapped BLAST can be utilized as described in Altschul et al, (1997) Nucleic Acids Res. 25(17):3389-3402. When utilizing BLAST and Gapped BLAST programs, the default parameters of the respective programs (e.g., XBLAST and NBLAST) can be used. See www.ncbi.nlm.nih.gov.

In other embodiments, the CDR amino acid sequences can be at least 90%, 91%, 92%, 93%, 94%, 95%, 96%, 97%, 98% or 99% identical to the respective sequences set forth above. In other embodiments, the amino acid sequences of the variable region can be at least 90%, 91%, 92%, 93%, 94%, 95%, 96%, 97%, 98% or 99% identical to the respective sequences set forth above.

Preferably, the CDRs of the isolated antibody or the antigen-binding portion thereof contain a conservative substitution of not more than 2 amino acids, or not more than 1 amino acid. The term “conservative substitution”, as used herein, refers to amino acid substitutions which would not disadvantageously affect or change the essential properties of a protein/polypeptide comprising the amino acid sequence. For example, a conservative substitution ma be introduced by standard techniques known in the art such as site-directed mutagenesis and PCR-mediated mutagenesis. Conservative amino acid substitutions include substitutions wherein an amino acid residue is substituted with another amino acid residue having a similar side chain, for example, a residue physically or functionally similar (such as, haying similar size, shape, charge, chemical property including the capability of forming covalent bond or hydrogen bond, etc.) to the corresponding amino acid residue. The families of amino acid residues haying similar side chains have been defined in the art. These families include amino acids having alkaline side chains (for example, Lysine, arginine and histidine), amino acids having acidic side chains (for example, aspartic acid and glutamic acid), amino acids having uncharged polar side chains (for example, glycine, asparagine, glutamine, serine, threonine, tyrosine, cysteine, tryptophan), amino acids having nonpolar side chains (for example, alanine, valine, leucine, isoleucine, proline, phenylalanine, methionine), amino acids having β-branched side chains (such as threonine, isoleucine) and amino acids having aromatic side chains (for example, tyrosine, phenylalanine, tryptophan, histidine). Therefore, a corresponding amino acid residue is preferably substituted with another amino acid residue from the same side-chain family. Methods for identifying amino acid conservative substitutions are well known in the art (see, for example, Brummell et al., Biochem. 32; 1180-1187 (1993); Kobayashi et al., Protein Eng. 12(10): 879-884 (1999); and Burks et al., Proc. Natl. Acad. Sci. USA 94; 412-417 (1997), which are incorporated herein by reference).

In certain embodiments, the first antigen-binding domain and the second antigen-binding domain of the bispecific antibody may be directly or indirectly connected to one another. In certain embodiments, the first antigen-binding domain and the second antigen-binding domain of the bispecific antibody may be connected to one another by a linker. In a specific embodiment, the linker is a peptide linker.

In certain embodiments, the first antigen-binding domain and the second antigen-binding domain of the bispecific antibody may be directly or indirectly connected to one another to form a bispecific antigen-binding molecule of the present invention (i.e. bispecific Scfv) further bound to an Fc region. Alternatively, the first antigen-binding, domain and the second antigen-binding domain may each be connected to a separate Fc region. Bispecific antigen-binding molecules of the present invention will typically comprise two Fc regions that are each individually part of a separate antibody heavy chain. The first and second Fc regions may be of the same sequence, except having a mutation in the C_{H}3 domain intended for the facilitation or ease of purification of heterodimeric (i.e. bispecific) molecules.

The Fc regions of the bispecific antibodies of the present invention may be human Fc regions. The Fc regions of the bispecific antibodies of the present invention may be of any isotype, including, but not limited to, IgG1, IgG2, IgG3 or IgG4. In one embodiment of this method, the Fc regions of both said first and said second antibodies are of the IgG1 isotype. In one embodiment of this method, the Fc regions of both said first and said second antibodies are of the IgG4 isotype. In another embodiment, one of the Fc regions of said antibodies is of the IgG1 isotype and the other of the IgG4 isotype. In the latter embodiment, the resulting bispecific antibody comprises an Fc region of an IgG1 and an Fc region of IgG4 and may thus have interesting intermediate properties with respect to activation of effector functions.

In the context of bispecific antibodies of the present invention, the Fe regions may comprise one or more amino acid changes (e.g., insertions, deletions or substitutions) as compared to the specified chimeric version of the Fc region, without changing the desired functionality. For example, the invention includes bispecific antigen-binding molecules comprising one or more modifications in the Fc region that results in a modified Fc region having a modified binding interaction (e.g., enhanced or diminished) between Fc and FcRn. Non-limiting examples of such Fc modifications include, e.g., a mutation of serine (“S”) to proline(“P”) at position 228 of the amino acid sequence of human IgG4 Fc region.

### Generation of Bispecific Antibodies

The bispecific antibodies and antigen-binding fragments provided herein can be made with any suitable methods known in the art. In a conventional approach, two immunoglobulin heavy chain-light chain pairs having different antigenic specificities can be co-expressed in a host cell to produce bispecific antibodies in a recombinant way (see, for example, Milstein and Cuello, Nature, 305: 537 (1983)), followed by purification by affinity chromatography.

Recombinant approach may also be used, where sequences encoding the antibody heavy chain variable domains for the two specificities are respectively fused to immunoglobulin constant domain sequences, followed by insertion to an expression vector which is co-transfected with an expression vector for the light chain sequences to a suitable host cell for recombinant expression of the bispecific antibody (see, for example, WO 94/04690; Suresh et al., Methods in Enzymology, 121:210 (1986)). Similarly, scFv dimers can also be recombinantly constructed and expressed from a host cell (see, e.g. Gruber et al., J. Immunol., 152:5368 (1994).)

In another method, leucine zipper, peptides from the Fos and Jun proteins can be linked to the Fab′ portions of two different antibodies by gene fusion. The linked antibodies are reduced at the hinge region to four half antibodies (i.e. monomers) and then re-oxidized to form heterodimers (Kostelny et al., J. Immunol., 148(5)1547-1553 (1992)).

The two antigen-binding domains may also be conjugated or cross-linked to form a bispecific antibody or antigen-binding fragment. For example, one antibody can be coupled to biotin while the other antibody to avidin, and the strong association between biotin and avidin would complex the two antibodies together to form a bispecific antibody (see, for example, U.S. Pat. No. 4,676,980 B2; WO 91/00360, WO 92/00373, and EP 03089). For another example, the two antibodies or antigen-binding fragments can be cross-linked by conventional methods known in the art, for example, as disclosed in U.S. Pat. No. 4,676,980 B2.

Bispecific antigen-binding fragments may be generated from a bispecific antibody, for example, by proteolytic cleavage, or by chemical linking. For example, an antigen-binding fragment (e.g. Fab⁵) of an antibody may be prepared and converted to Fab′-thiol derivative and then mixed and reacted with another converted Fab⁵ derivative having a different antigenic specificity to form a bispecific antigen binding: fragment (see, for example, Brennan et al., Science, 229: 81 (1985)).

In certain embodiments, the bispecific antibody or antigen-binding fragments may be engineered at the interface so that a knob-into-hole association can be formed to promote heterodimerization of the two different antigen-binding sites. “Knob-into-hole” as used herein, refers to an interaction between two polypeptides (such as CH3 domain), where one polypeptide has a protuberance (i.e. “knob”) due to presence of an amino acid residue having a bulky side chain (e.g. tyrosine or tryptophan), and the other polypeptide has a cavity (i.e. “hole”) where a small side chain amino acid residue resides (e.g. alanine threonine), and the protuberance is positionable in the cavity so as to promote interaction of the two polypeptides to form a heterodimer or a complex. Methods of generating, polypeptides with knobs-into-holes are known in the art, e.g., as described in U.S. Pat. No. 5,731,168B2.

### Nucleic Acid Molecules Encoding Antibodies of the Invention

In some aspects, the invention is directed to an isolated nucleic acid molecule, comprising a nucleic acid sequence encoding the heavy chain variable region and/or the light chain variable region of the bispecific antibody as disclosed herein.

in some aspects, the invention is directed to a vector comprising the nucleic acid sequence encoding the heavy chain variable region and/or the light chain variable region of the bispecific antibody as disclosed herein. In a further embodiment, the expression vector further comprises a nucleotide sequence encoding the constant region of a light chain, a heavy chain or both light and heavy chains of a bispecific antibody, e.g. a humanized bispecific antibody.

A vector in the context of the present invention may be any suitable vector, including chromosomal, non-chromosomal, and synthetic nucleic acid vectors (a nucleic acid sequence comprising, a suitable set of expression control elements). Examples of such vectors include derivatives of SV40 , bacterial plasmids, phage DNA, baculovirus, yeast plasmids, vectors derived from combinations of plasmids and phage DNA, and viral nucleic acid (RNA or DNA) vectors. In one embodiment, a CD20 or a CD3 antibody-encoding nucleic acid is comprised in a naked DNA or RNA vector, including, for example, a linear expression element (as described in for instance Sykes and Johnston, Nat Biotech 17, 355-59 (1997)), a compacted nucleic acid vector (as described in for instance U.S. Pat. No. 6,077, 835 and/or WO 00/70087), a plasmid vector such as pBR322, puC, 19/18, or pUC 118/119, a “midge” minimally-sized nucleic acid vector (as described in for instance Schakowski et al. , Mol Ther 3, 793-800 (2001)), or as a precipitated nucleic acid vector construct, such as a CaP04-precipitated construct (as described in for instance WO2000416147, Benvenisty and Reshef, PNAS USA 83, 9551-55 (1986), Wigler et al., Cell 14, 725 (1978), and Coraro and Pearson, Somatic Cell Genetics 7, 603 (1981)). Such nucleic acid vectors and the usage thereof are well known in the art (see for instance U.S. Pat. Nos. 5,589,466 and 5,973,972).

In one embodiment, the vector is suitable for expression of the CD20 antibody €nd/or the CD3 antibody in a bacterial cell. Examples of such vectors include expression vectors such as BlueScript (Stratagene), pIN vectors (Van Heeke Schuster, J Blot Chem 264, 5503-5509 (1989), pET vectors (Novagen, Madison Wis.) and the like). A vector may also or alternatively be a vector suitable for expression in a yeast system. Any vector suitable for expression in a yeast system may be employed. Suitable vectors include, for example, vectors comprising constitutive or inducible promoters such as alpha factor, alcohol oxidase and PGH (reviewed in: F. Ausubel et al., ed. Current Protocols in Molecular Biology, Greene Publishing and Wiley InterScience New York (1987), and Grant et al., Methods in Enzymol 153, 516-544 (1987)).

A vector may also or alternatively be a vector suitable for expression in mammalian cells, e.g. a vector comprising glutamine symhetase as a selectable marker, such as the vectors described in Bebbington (1992) Biotechnology (NY) 10: 169-175.

A nucleic acid and/or vector may also comprise a nucleic acid sequence encoding a secretion/localization sequence, which can target a polypeptide, such as a nascent polypeptide chain, to the periplasmic space or into cell culture media. Such sequences are known in the art, and include secretion leader or signal peptides.

The vector may comprise or be associated with any suitable promoter, enhancer, and other expression-facilitating elements. Examples of such elements include strong expression promoters (e. g., human CMV IE promoter/enhancer as well as RSV, SV40, SL3-3, MMTV, and HIV LTR promoters), effective poly (A) termination sequences, an origin of replication for plasmid product in E. coli, an antibiotic resistance gene as selectable marker, and/or a convenient cloning site (e.g., a polylinker). Nucleic acids may also comprise an inducible promoter as opposed to a constitutive promoter such as CMV IE.

In an even further aspect, the invention relates to a host cell comprising the vector specified herein above.

Thus the present invention also relates to a recombinant eukaryotic or prokaryotic host cell which produces a bispecific antibody of the present invention, such as a transfectoma.

The CD20-specific antibody may be expressed in a recombinant eukaryotic or prokaryotic host cell, such as a transfectoma, which produces an antibody of the invention as defined herein or a bispecific antibody of the invention as defined herein. The CD3- specific antibody may likewise be expressed in a recombinant eukaryotic or prokaryotic host cell, such as a transfectoma, which produces an antibody of the invention as defined herein or a bispecific antibody of the invention as defined herein.

Examples of host cells include yeast, bacterial, plant and mammalian cells, such as CHO, CHO-S, HEK, HEK293, HEK-293F, Expi293F, PER.C6 or NSO cells or lymphocytic cells. For example, in one embodiment, the host cell may comprise a first and second nucleic acid construct stably integrated into the cellular genome. In another embodiment, the present invention provides a cell comprising a non-integrated nucleic acid, such as a plasmid, cosmid, phagemid, or linear expression element, which comprises a first and second nucleic acid construct as specified above.

In an even further aspect, the invention relates to a transgenic non-human animal or plant comprising nucleic acids encoding one or two sets of a human heavy chain and a human light chain, wherein the animal or plant produces a bispecific antibody of the invention.

In a further aspect, the invention relates to a hybridoma which produces an antibody for use in a bispecific antibody of the invention as defined herein. In an even further aspect, the invention relates to a transgenic non-human animal or plant comprising nucleic acids encoding one or two sets of a human heavy chain and a human light chain, wherein the animal or plant produces an antibody for use in a bispecific antibody or a bispecific antibody of the invention.

In one aspect, the invention relates to an expression vector comprising

(i) a nucleic acid sequence encoding a heavy chain sequence of a first antigen-binding site according to any one of the embodiments disclosed herein;

(ii) a nucleic acid sequence encoding a light chain sequence of a first antigen-binding site according to any one of the embodiments disclosed herein;

(iii) a nucleic acid sequence encoding a heavy chain sequence of a second antigen-binding site according to any one of the embodiments disclosed herein;

(iv) a nucleic acid sequence encoding a light chain sequence of a second antigen-binding site according to any one of the of the embodiments disclosed herein;

(v) the nucleic acid set forth in (i) and the nucleic acid set forth in (ii);

(vi) the nucleic acid set forth in (iii) and the nucleic acid set forth in (iv).

(vii) the nucleic acid set forth in (i), (ii), (iii) and (iv).

In one aspect, the invention relates to a nucleic acid construct encoding one or more amino acid sequences set out in the sequence listing.

In one aspect, the invention relates to a method for producing a bispecific antibody according to any one of the embodiments as disclosed herein, comprising the steps of culturing a host cell as disclosed herein comprising an expression vector or more than one expression vectors as disclosed herein expressing the bispecific antibody as disclosed herein and purifying said antibody from the culture media. In one aspect, the invention relates to a host cell comprising an expression vector as defined above. In one embodiment, the host cell is a recombinant eukaryotic, recombinant prokaryotic, or recombinant microbial host cell.

### Pharmaceutical Compositions

In some aspects, the invention is directed to a pharmaceutical composition comprising at least one antibody or antigen-binding portion thereof as disclosed herein and a pharmaceutically acceptable carrier.

### Components of the Compositions

The pharmaceutical composition may optionally contain one or more additional pharmaceutically active ingredients, such as another antibody or a drug. The pharmaceutical compositions of the invention also can be administered in a combination therapy with, for example, another immune-stimulatory agent, anti-cancer agent, an antiviral agent, or a vaccine, such that the anti-CD³/anti-CD20 bispecific antibody enhances the immune response against the vaccine. A pharmaceutically acceptable carrier can include, for example, a pharmaceutically acceptable liquid, gel or solid carriers, an aqueous medium, a non-aqueous medium, an anti-microbial agent, isotonic agents, buffers, antioxidants, anesthetics, suspending/dispersing agent, a chelating agent, a diluent, adjuvant, excipient or a nontoxic auxiliary substance, other known in the art various combinations of components or more.

Suitable components may include, for example, antioxidants, fillers, binders, disintegrating agents, buffers, preservatives, lubricants, flavorings, thickening agents, coloring agents, emulsifiers or stabilizers such as sugars and cyclodextrin. Suitable anti-oxidants may include, for example, methionine, ascorbic acid, EDTA, sodium thiosulfate, platinum, catalase, citric acid, cysteine, mercapto glycerol, thioglycolic acid, Mercapto sorbitol, butyl methyl anisole, butylated hydroxy toluene and/or propylgalacte. As disclosed in the present invention, in a solvent containing an antibody or an antigen-binding fragment of the present invention discloses compositions include one or more anti-oxidants such as methionine, reducing antibody or antigen binding fragment thereof may be oxidized. The oxidation reduction may prevent or reduce a decrease in binding affinity, thereby enhancing antibody stability and extended shelf life. Thus, in some embodiments, the present invention provides a composition comprising one or more antibodies or antigen binding fragment thereof and one or more anti-oxidants such as methionine. The present invention further provides a variety of methods, wherein an antibody or antigen binding fragment thereof is mixed with one or more anti-oxidants, such as methionine, so that the antibody or antigen binding fragment thereof can be prevented from oxidation, to extend their shelf life and/or increased activity.

To further illustrate, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcelluose, hydroxypropyl methylcellulose., or polyvinylpyrrolidone, emulsifying agents such as Polysorbate 80 (TWEEN-80), sequestering or chelating agents such as EDTA (ethylenediaminetenaacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

### Administration, Formulation and Dosage

The pharmaceutical composition of the invention may be administered in vivo, to a subject in need thereof, by various routes, including, but not limited to, oral, intravenous, intra-arterial, subcutaneous, parenteral, intranasal, intramuscular, intracranial, intracardiac, intraventricular, intratracheal, buccal, rectal, intraperitoneal, intradermal, topical, transdermal, and intrathecal, or otherwise by implantation or inhalation. The subject compositions may be formulated into preparations in solid, semi-solid, liquid, or gaseous forms; including, but not limited to, tablets, capsules, powders, granules, ointments, solutions, suppositories, enemas, injections, inhalants, and aerosols. The appropriate formulation and route of administration may be selected according to the intended application and therapeutic regimen.

Suitable formulations for enteral administration include hard or soft gelatin capsules, pills, tablets, including coated tablets, elixirs, suspensions, syrups or inhalations and controlled release forms thereof.

Formulations suitable for parenteral administration (e.g., by injection), include aqueous or non-aqueous, isotonic, pyrogen-free, sterile liquids (e.g., solutions, suspensions), in which the active ingredient is dissolved, suspended, or otherwise provided (e.g., in a liposome or other microparticulate). Such liquids may additional contain other pharmaceutically acceptable ingredients, such as anti-oxidants, buffers, preservatives, stabilisers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection. Similarly, the particular dosage regimen, including dose, timing and repetition, will depend on the particular individual and that individual's medical history, as well as empirical considerations such as pharmacokinetics (e.g., half-life, clearance rate, etc.).

Frequency of administration may be determined and adjusted over the course of therapy, and is based on reducing the number of proliferative or tumorigenic cells, maintaining the reduction of such neoplastic cells, reducing the proliferation of neoplastic cells, or delaying the development of metastasis. In some embodiments, the dosage administered may be adjusted or attenuated to manage potential side effects and/or toxicity. Alternatively, sustained continuous release formulations of a subject therapeutic composition may be appropriate.

It will be appreciated by one of skill in the art that appropriate dosages can vary from patient to patient. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the patient. The amount of compound and route of administration will ultimately be at the discretion of the physician, veterinarian, or clinician, although generally the dosage will be selected to achieve local concentrations at the site of action that achieve the desired effect without causing substantial harmful or deleterious side-effects.

In general, the antibody or the antigen binding portion thereof of the invention may be administered in various ranges. These include about 5 μg/kg, body weight to about 100 mg/kg body weight per dose; about 50 μg/kg body weight to about 5 mg/kg body weight per dose; about 100 μg/kg body weight to about 10 mg/kg body weight per dose. Other ranges include about 100 μg/kg body weight to about 20 mg/kg body weight per dose and about 0.5 mg/kg body weight to about 20 mg/kg body weight per dose. In certain embodiments, the dosage is at least about 100 μg/kg body weight, at least about 250 μg/kg body weight, at least about 750 μg/kg body weight, at least about 3 mg/kg body weight, at least about 5 mg/kg body weight, at least about 10 mg/kg body weight.

In any event, the antibody or the antigen binding portion thereof of the invention is preferably administered as needed to subjects in need thereof. Determination of the frequency of administration may be made by persons skilled in the art, such as an attending physician based on considerations of the condition being treated, age of the subject being treated, severity of the condition being treated, general state of health of the subject being treated and the like.

In certain preferred embodiments, the course of treatment involving the antibody or the antigen-binding portion thereof of the instant invention will comprise multiple doses of the selected drug product over a period of weeks or months. More specifically, the antibody or the antigen binding portion thereof of the instant invention may be administered once every day, every two days, every four days, every week, every ten days, every two weeks, every three weeks, every month, every six weeks, every two months, every ten weeks or every three months. In this regard, it will be appreciated that the dosages may be altered or the interval may be adjusted based on patient response and clinical practices.

Dosages and regimens may also be determined empirically for the disclosed therapeutic compositions in individuals who have been given one or more administration(s). For example, individuals may be given incremental dosages of a therapeutic composition produced as described herein. In selected embodiments, the dosage may be gradually increased or reduced or attenuated based respectively on empirically determined or observed side effects or toxicity. To assess efficacy of the selected composition, a marker of the specific disease, disorder or condition can be followed as described previously. For cancer, these include direct measurements of tumor site via palpation or visual observation, indirect measurement of tumor size by x-ray or other imaging techniques; an improvement as assessed by direct tumor biopsy and microscopic examination of the tumor sample; the measurement of an indirect tumor marker (e.g., PSA for prostate cancer) or a tumorigenic antigen identified according to the methods described herein, a decrease in pain or paralysis; improved speech, vision, breathing or other disability associated with the tumor; increased appetite; or an increase in quality of life as measured by accepted tests or prolongation of survival. It will be apparent to one of skill in the art that the dosage will vary depending on the individual, the type of neoplastic condition, the stage of neoplastic condition, whether the neoplastic condition has begun to metastasize to other location in the individual, and the past and concurrent treatments being used.

Compatible formulations for parenteral administration (e.g., intravenous injection) will comprise the antibody or antigen-binding portion thereof as disclosed herein in concentrations of from about 10 μg/ml to about 100 μg/ml. In certain selected embodiments, the concentrations of the antibody or the antigen binding portion thereof will comprise 20 μg/ml, 40 μg/ml, 60 μg/ml, 80 μg/ml, 100 μg/ml, 200 μg/ml, 300, μg/ml, 400 μg/ml, 500 μg/ml, 600 μg/ml, 700 μg/ml, 800 μg/ml , 900 μg/ml or 1 mg/ml. In other preferred embodiments ADC concentrations will comprise 2 mg/ml, 3 mg/ml, 4 mg/ml, 5 mg/ml, 6 mg/ml, 8 mg/ml, 10 mg/ml, 12 mg/ml, 14 mg/ml, 16 mg/ml, 18 mg/ml, 20 mg/ml, 25 mg/ml, 30 mg/ml, 35 mg/ml, 40 mg/ml, 45 mg/ml, 50 mg/ml, 60 mg/ml, 70 mg/ml, 80 mg/ml, 90 mg/ml or 100 mg/ml.

### Applications of the Invention

In some aspects, the present invention provides a method of treating a disorder in a subject, which comprises administering to the subject (for example, a human) in need of treatment a therapeutically effective amount of the antibody or antigen-binding portion thereof as disclosed herein. For example, the disorder is a cancer.

A variety of cancers where CD3 and/or CD20 is implicated, whether malignant or benign and whether primary or secondary, may be treated or prevented with a method provided by the disclosure. The cancers may be solid cancers or hematologic malignancies. Examples of such cancers include lung cancers such as bronchogenic carcinoma (e.g., squamous cell carcinoma, small cell carcinoma, large cell carcinoma, and adenocarcinoma), alveolar cell carcinoma, bronchial adenoma, chondromatous hamartoma (noncancerous), and sarcoma (cancerous); heart cancer such as myxoma, fibromas, and rhabdomyomas; bone cancers such as osteochondromas, condromas, chondroblastomas, chondromyxoid fibromas, osteoid osteo tans, giant cell tumors, chondrosarcoma, multiple myeloma, osteosarcoma, fibrosarcomas, malignant fibrous histiocytomas, Ewing's tumor (Ewing's sarcoma), and reticulum cell sarcoma; brain cancer such as gliomas (e.g., glioblastoma mulltiforme), anaplastic astrocytomas, astrocytomas, oligodendrogliomas, medulloblastomas, chordoma, Schwannomas, ependymomas, meningiomas, pituitary adenoma, pinealoma, osteomas, hemangioblastomas, craniopharyngiomas, chordomas, germinomas, teratomas, dermoid cysts, and angiomas; cancers in digestive system such as colon cancer, leiomyoma, epidermoid carcinoma, adenocarcinoma, leiomyosarcoma, stomach adenocarcinomas, intestinal lipomas, intestinal neurolibromas, intestinal fibromas, polyps in large intestine, and colorectal cancers; liver cancers such as hepatocellular adenomas, hemangioma, hepatocellular carcinoma, fibrolamellar carcinoma, cholangiocarcinoma, hepatoblastoma, and angiosarcoma; kidney cancers such as kidney adenocarcinoma, renal cell carcinoma, hypernephroma, and transitional cell carcinoma of the renal pelvis; bladder cancers; hematological cancers such as acute lymphocytic (lymphoblastic) leukemia, acute myeloid (myelocytic, myelogenous, myeloblasts, myelomonocytic) leukemia, chronic lymphocytic leukemia (e.g., Sezary syndrome and hairy cell leukemia), chronic myelocytic (myeloid, myelogenous, granulocytic) leukemia, Hodgkin's lymphoma, non-Hodgkin's lymphoma, B cell lymphoma, mycosis fungoides, and myeloproliferative disorders (including myeloproliferative disorders such as polycythemia vera, myelalibrosis, thrombocythemia, and chronic myelocytic leukemia); skin cancers such as basal cell Carcinoma, squamous cell carcinoma, melanoma, Kaposi's sarcoma, and Paget's disease; head and neck cancers; eye-related cancers such as retinoblastoma and intraoccular melanocarcinoma; male reproductive system cancers such as benign prostatic hyperplasia, prostate cancer, and testicular cancers (e.g., seminoma, teratoma, embryonal carcinoma, and choriocarcinoma); breast cancer; female reproductive system cancers such as uterine cancer endometrial carcinoma), cervical cancer (cervical carcinoma), cancer of the ovaries (ovarian carcinoma), vulvar carcinoma, vaginal carcinoma, fallopian tube cancer, and hydatidiform mole; thyroid cancer (including papillary, follicular, anaplastic, or medullary cancer); pheochromocytomas (adrenal gland); noncancerous growths of the parathyroid glands; pancreatic cancers; and hematological cancers such as leukemias, myelomas, non-Hodgkin's lymphomas, and Hodgkin's lymphomas. In a specific embodiment, the cancer is colon cancer.

In some embodiments, examples of cancer include but not limited to B-cell cancers, including B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; and post-transplant lymphoproblerative disorder (PTLD), as well as abnormal vascular proliferation associated with phakomatoses, edema (such as that associated with brain tumors), B-cell proliferative disorders, and Meigs' syndrome. More specific examples include, but are not limited to, relapsed or refractory NHL, front line low grade NHL, Stage III/IV NHL, chemotherapy resistant NHL, precursor B lymphoblastic leukemia and/or lymphoma, small lymphocytic lymphoma, B-cell chronic lymphocytic leukemia and/or prolymphocytic leukemia and/or small lymphocytic lymphoma, B-cell prolymphocytic lymphoma, immunocytoma and/or lymphoplasmacytic lymphoma, lymphoplasmacytic lymphoma, marginal zone B-cell lymphoma, splenic marginal zone lymphoma, extranodal marginal zone-MALT lymphoma, nodal marginal zone lymphoma, hairy cell leukemia, plasmacytoma and/or plasma cell myeloma, low grade/follicular lymphoma, intermediate grade/follicular NHL, mantle cell lymphoma, follicle center lymphoma (follicular), intermediate grade diffuse NHL, diffuse large B-cell lymphoma, aggressive NHL (including aggressive front-line NHL and aggressive relapsed NHL), NHL relapsing after or refractory to autologous stem cell transplantation, primary mediastinal large B-cell lymphoma, primary effusion lymphoma, high grade immunoblastic NHL, high grade lymphoblastic NHL, high grade small non-cleaved cell NHL, bulky disease NHL, Burkitt's lymphoma, precursor (peripheral) large granular lymphocytic leukemia, mycosis fungoides and or Sezary syndrome, skin (cutaneous) lymphomas, anaplastic large cell lymphoma, angiocentric lymphoma.

In some embodiments, examples of cancer further include, but are not limited to, B-cell proliferative disorders, which further include, but are not limited to, lymphomas (e.g., B-Cell Non-Hodgkin's lymphomas (NHL)) and lymphocytic leukemias. Such lymphomas and lymphocytic leukemias include e.g. a) follicular lymphomas, b) Small Non-Cleaved Cell Lymphomas/Burkitt's lymphoma (including endemic Burkitt's lymphoma, sporadic Burkitt's lymphoma and Non-Burkitt's lymphoma), c) marginal zone lymphomas (including extranodal marginal one B-cell lymphoma (Mucosa-associated lymphatic tissue lymphomas, MALT), nodal marginal zone B-cell lymphoma and splenic marginal zone lymphoma), d) Mantle cell lymphoma (MCL), e) Large Cell Lymphoma (including, diffuse large cell lymphoma (DLCL), Diffuse Mixed Cell Lymphoma, Immunoblastic Lymphoma, Primary Mediastinal B-Cell Lymphoma, Angiocentric Lymphoma, Pulmonary B-Cell Lymphoma), f) hairy cell leukemia, g) lymphocytic lymphoma, Waldenstrom's macroglobulinemia, h) acute lymphocytic leukemia (ALL), chronic lymphocytic leukemia (CLL)/small lymphocytic lymphoma (SLL), B cell prolymphocytic leukemia, i) plasma cell neoplasms, plasma cell myeloma, multiple myeloma, plasmacytoma, and/or j) Hodgkin's disease.

In some other embodiments, the disorder is an autoimmune disease. Examples of autoimmune diseases that may be treated with the antibody or antigen-bindings portion thereof include autoimmune encephalomyelitis, lupus erythematosus, and rheumatoid arthritis. The antibody or the antigen-binding portion thereof may also be used to treat or prevent infections disease, inflammatory disease such as allergic asthma) and chronic graft-versus-host disease.

### Combined Use With Chemotherapies

The antibody or the antigen-binding portion thereof may be used in combination with an anti-cancer agent, a cytotoxic agent or chemotherapeutic agent.

The term “anti-cancer agent” or “anti-proliferative agent” means any agent that can be used to treat a cell proliferative disorder such as cancer, and includes, but is not limited to, cytotoxic agents, cytostatic agents, anti-angiogenic agents, debulking agents, chemotherapeutic agents, radiotherapy and radiotherapeutic agents, targeted anti-cancer agents, BRMs, therapeutic antibodies, cancer vaccines, cytokines, hormone therapies, radiation therapy and anti-metastatic agents and immunotherapeutic agents. It will be appreciated that, in selected embodiments as discussed above, such anti-cancer agents may comprise conjugates and may be associated with the disclosed site-specific antibodies prior to administration. More specifically, in certain embodiments selected anti-cancer agents will be linked to the unpaired cysteines of the engineered antibodies to provide engineered conjugates as set forth herein. Accordingly, such engineered conjugates are expressly contemplated as being within the scope of the instant invention. In other embodiments, the disclosed anti-cancer agents will be given in combination with site-specific conjugates comprising a different therapeutic agent as set forth above.

As used herein the term “cytotoxic agent.” means a substance that is toxic to the cells and decreases or inhibits the function of cells and/or causes destruction of cells. In certain embodiments, the substance is a naturally occurring molecule derived from a living organism. Examples of cytotoxic agents include, but are not limited to, small molecule toxins or enzymatically active toxins of bacteria (e.g., Diptheria toxin, Pseudomonas endotoxin and exotoxin, Staphylococcal enterotoxin A), fungal (e.g., α-sarcin, restrictocin), plants (e.g., abrin, ricin, modeccin, viscumin, pokeweed anti-viral protein, saporin, gelonin, momoridin, trichosarithin, barley toxin, Aleurites fordii proteins, dianthin proteins, Phytolacca mericana proteins (PAPI, PAHII, and PAP-S), Momordica charantia inhibitor, curcin, crotin, saponaria officinalis inhibitor, gelonin, mitegellin, restrictocin, phenomycin, neomycin, and the tricothecenes) or animals, (e.g., cytotoxic Rases, such as extracellular pancreatic RNases: DNase I, including fragments and/or variants thereof).

For the purposes of the instant invention a “chemotherapeutic agent” comprises a chemical compound that non-specifically decreases or inhibits the growth, proliferation, and/or survival of cancer cells (e.g., cytotoxic or cytostatic agents). Such chemical agents are often directed to intracellular processes necessary for cell growth or division, and are thus particularly effective against cancerous cells, which generally grow and divide rapidly. For example, vincristine polymerizes microtubules, and thus inhibits cells from entering mitosis. In general, chemotherapeutic agents can include any chemical agent that inhibits, or is designed to inhibit, a cancerous cell or a cell likely to become cancerous or generate tumorigenic progeny (e.g., TIC). Such agents are often administered, and are often most effective, in combination, e.g., in regimens such as CHOP or FOLFIRI.

Examples of anti-cancer agents that may be used in combination with the site-specific; constructs of the present invention (either as a component of a site specific conjugate or in an unconjugated state) include, but are not limited to, alkylating agents, alkyl sulfonates, aziridines, ethylenimines and methylamelamines, acetocenins, a camptothecin, bryostatin, callystatin, CC-106:5, cryptophycins, dolastatin, duocarmycin, eleutherobin, pancratistatin, a sarcodictyin, spongistatin, nitrogen mustards, antibiotics, enediyne antibiotics, dynemicin, hisphosphonates, esperamicin, chromoprotein enediyne antiobiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleticine, ADRIAMYCIN® doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonicrin, streptozocin, tubercidin, ubenimex, zinostatin, zorubicin; anti-metabolites, erlotinib, vemurafenib, crizotinib,sorafenib, ibrutinib, enzalutamide, folic acid analogues, purine analogs, androgens, anti-adrenals, folic acid replenisher such as frolinic acid, aceglatone, aidophosphamide glycoside, aminolevulinic acid, eniluracil, amsacrine, bestrabucil, bisantrene, edatraxate, defofamine, demecolcine, diaziquone, elfornithine, elliptinium acetate, an epothilone, etoglucid, gallium nitrate, hydroxymea, lentinan, lonidainine, maytansitioids, mitoguazone, mitoxantrone, mopidanmol, nitraerine, pentostatin, phenamet, pirarubicin, losoxantrone, podophyllinic acid, 2-ethylhydrazide, procarbazine, PSK®polysaccharide complex (JHS Natural Products, Eugene, Oreg.), razoxane; rhizosin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; marmornustine; mitobronitol; mitolactol; pipobroman; gacytosine; arabinoside (“Ara-C”); cyclophosphamide; thiotepa; taxoids, chloranbucil; GEMZAR® gemcitabine; 6-thioguanine; mercaptopurine: methotrexate, platinum analogs, vinblastine; platinum; etoposide (VP-16); ifosfatnide; mitoxantrone; vincristine; NAVELBINE® vinorelbine: novantrone; teniposide; edatrexate; daunomycin; aminopterin; xeloda; ibandronate; irinotecan (Camptosar, CPT-11), topoisomerase inhibitor RFS 2000; difluoromethylornithine; retinoids; capecitabine; combretastatin; leucovorin; oxaliplain; inhibitors of PKC-alpha, Raf, H-Ras, EGFR and VEGE-A that reduce cell proliferation and pharmaceutically acceptable salts, acids or derivatives of any of the above. Also included in this definition are anti-hormonal acents that act to regulate or inhibit hormone action on tumors such as anti-estrogens and selective estrogen receptor modulators, aromatase inhibitors that inhibit the enzyme aromatase, which regulates estrogen production in the adrenal glands, and anti-androgens; as well as troxacitabine (a 1,3-dioxolane nucleoside cytosine analog); antisense oligonucleotides, ribozymes such as a VEGF expression inhibitor and a HER2 expression inhibitor; vaccines, PROLEUKIN® rIL-2; LURTOTECAN® topoisomerase 1 inhibitor; ABARELIX® rmRH; Vinorelbine and Esperamicins and pharmaceutically acceptable salts, acids or derivatives of any of the above.

### Combined Use With Radiotherapies

The present invention also provides for the combination of the antibody or the antigen-binding portion thereof with radiotherapy (i.e., any mechanism for inducing DNA damage locally within tumor cells such as gamma-irradiation, X-rays, UV-irradiation, microwaves, electronic emissions and the like). Combination therapy using the directed delivery of radioisotopes to tumor cells is also contemplated, and the disclosed conjugates may be used in connection with a targeted anti-cancer agent or other targeting means. Typically, radiation therapy is administered in pulses over a period of time from about 1 to about 2 weeks. The radiation therapy may be administered to subjects having head and neck cancer for about 6 to 7 weeks. Optionally, the radiation therapy may be administered as a single dose or as multiple, sequential doses.

### Pharmaceutical Packs and Kits

Pharmaceutical packs and kits comprising one or more containers, comprising one or more doses of the antibody or the antigen-binding portion thereof are also provided. In certain embodiments, a unit dosage is provided wherein the unit dosage contains a predetermined amount of a composition comprising, for example, the antibody or the antigen-binding portion thereof, with or without one or more additional agents. For other embodiments, such a unit dosage is supplied in single-use prefilled syringe for injection. In still other embodiments, the composition contained in the unit dosage may comprise saline, sucrose, or the like; a buffer, such as phosphate, or the like; and/or be formulated within a stable and effective pH range. Alternatively, in certain embodiments, the conjugate composition may be provided as a lyophilized powder that may be reconstituted upon addition of an appropriate liquid, for example, sterile water or saline solution. In certain preferred embodiments, the composition comprises one or more substances that inhibit protein aggregation, including, but not limited to, sucrose and arginine. Any label on, or associated with, the container(s) indicates that the enclosed conjugate composition is used for treating the neoplastic disease condition of choice.

The present invention also provides kits for producing single-dose or multi-dose administration units of site-specific conjugates and, optionally, one or more anti-cancer agents. The kit comprises a container and a label or package insert on or associated with the container. Suitable containers include, for example, bottles, vials, syringes, etc. The containers may be formed from a variety of materials such as glass or plastic and contain a pharmaceutically effective amount of the disclosed conjugates in a conjugated or unconjugated form. In other preferred embodiments, the container(s) comprise a sterile access port (for example the container may be an intravenous solution bag or a vial having a stopper pierceable by a hypodermic injection needle). Such kits will generally contain in a suitable container a pharmaceutically acceptable formulation of the engineered conjugate and, optionally, one or more anti-cancer agents in the same or different containers. The kits may also contain other pharmaceutically acceptable formulations, either for diagnosis or combined therapy. For example, in addition to the antibody or the antigen-binding portion thereof of the invention such kits may contain any one or more of a range of anti-cancer agents such as chemotherapeutic or radiotherapeutic drugs; anti-angiogenic agents; anti-metastatic agents targeted anti-cancer agents; cytotoxic agents; and/or other anti-cancer agents.

More specifically the kits may have a single container that contains the disclosed the antibody or the antigen-binding portion thereof, with or without additional components, or they may have distinct containers for each desired agent. Where combined therapeutics are provided for conjugation, a single solution may be pre-mixed, either in a molar equivalent combination, or with one component in excess of the other. Alternatively, the conjugates and any optional anti-cancer agent of the kit may be maintained separately within distinct containers prior to administration to a patient. The kits may also comprise a second/third container means for containing a sterile, pharmaceutically acceptable buffer or other diluent such as bacteriostatic water for injection (BWFI), phosphate-buffered saline (PBS), Ringer's solution and dextrose solution.

When the components of the kit are provided in one or more liquid solutions, the liquid solution is preferably an aqueous solution, with a sterile aqueous or saline solution being particularly preferred. However, the components of the kit may be provided as dried powder(s). When reagents or components are provided as a dry powder, the powder can be reconstituted by the addition of a suitable solvent. It is envisioned that the solvent may also be provided in another container.

As indicated briefly above the kits may also contain a means by which to administer the antibody or the antigen-binding portion thereof and any optional components to a patient, e.g., one or more needles, I.V. bags or syringes, or even an eye dropper, pipette, or other such like apparatus, from which the formulation may be injected or introduced into the animal or applied to a diseased area of the body. The kits of the present invention will also typically include a means for containing the vials, or such like, and other component in close confinement for commercial sale, such as, e.g., injection or blow-molded plastic containers into which the desired vials and other apparatus are placed and retained.

### Sequence Listing Summary

Appended to the instant application is a sequence listing comprising a number of nucleic acid and amino acid sequences. The following Table A provides a summary of the included sequences.

The illustrative antibody as disclosed herein, which is humanized anti-CD3/anti-CD20 bispecific antibody, is designated as “T3U3-E4-1.uIgG4.SP”. The bispecific antibody has two antigen-binding site, one specifically binds to CD3, and the other specifically binds to CD20. The antigen-binding site that specifically binds to CD3 is also referred to “anti-CD3 arm”, and the antigen-binding site that specifically binds to CD20 is also referred to “anti-CD20 arm”.

Specifically, in the bispecific antibody “T3U3-E4-1.uIgG4.SP”, the “anti-CD3 arm” (SEQ ID NO: 37) and the “anti-CD20 arm” (SEQ ID NO: 38) each is linked by a hinge sequence (SEQ ID NO: 41) to human IgG4 Fc region (SEQ ID NO: 42), wherein the human IgG4 contains a S228P mutation which is present in the hinge sequence (SEQ ID NO: 41).

TABLE A

| SEQ ID NO. | Description |
|---|---|
| 1 | CDR1 in the heavy chain variable region (“VH”) of “anti-CD3 arm” |
| 2 | CDR2 in the heavy chain variable region of “anti-CD3 arm” |
| 3 | CDR3 in the heavy chain variable region of “anti-CD3 arm” |
| 4 | CDR1 in the light chain variable region (“VL”) of “anti-CD3 arm” |
| 5 | CDR2 in the light chain variable region of “anti-CD3 arm” |
| 6 | CDR3 in the light chain variable region of “anti-CD3 arm” |
| 7 | CDR1 in the heavy chain variable region of “anti-CD20 arm” |
| 8 | CDR2 in the heavy chain variable region of “anti-CD20 arm” |
| 9 | CDR3 in the heavy chain variable region of “anti-CD20 arm” |
| 10 | CDR1 in the light chain variable region of “anti-CD20 arm” |
| 11 | CDR2 in the light chain variable region of “anti-CD20 arm” |
| 12 | CDR3 in the light chain variable region of “anti-CD20 arm” |
| 13 | Amino acid sequence of VH of “anti-CD3 arm” |
| 14 | Amino acid sequence of VL of “anti”CD3 arm” |
| 15 | Amino acid sequence of VH of “anti-CD20 arm” |
| 16 | Amino acid sequence of VL of “anti-CD20 arm” |
| 17 | DNA sequence encoding VH of “anti-CD3 arm” |
| 18 | DNA sequence encoding VL of “anti-CD3 arm” |
| 19 | DNA sequence encoding VH of “anti-CD20 arm” |
| 20 | DNA sequence encoding VL of “anti-CD20 arm” |
| 21 | FW1 in the heavy chain variable region of “anti-CD3 arm” |
| 22 | FW2 in the heavy chain variable region of “anti-CD3 arm” |
| 23 | FW3 in the heavy chain variable region of “anti-CD3 arm” |
| 24 | FW4 in the heavy chain variable region of “anti-CD3 arm” |
| 25 | FW1 in the light chain variable region of “anti-CD3 arm” |
| 26 | FW2 in the light chain variable region of “anti-CD3 arm” |
| 27 | FW3 in the light chain variable region of “anti-CD3 arm” |
| 28 | FW4 in the light chain variable region of “anti-CD3 arm” |
| 29 | FW1 in the heavy chain variable region of “anti-CD20 arm” |
| 30 | FW2 in the heavy chain variable region of “anti-CD20 arm” |
| 31 | FW3 in the heavy chain variable region of “anti-CD20 arm” |
| 32 | FW4 in the heavy chain variable region of “anti-CD20 arm” |
| 33 | FW1 in the light chain variable region of “anti-CD20 arm” |
| 34 | FW2 in the light chain variable region of “anti-CD20 arm” |
| 35 | FW3 in the light chain variable region of “anti-CD20 arm” |
| 36 | FW4 in the light chain variable region of “anti-CD20 arm” |
| 37 | Full-length amino acid sequence of “anti-CD3 arm” |
| 38 | Full-length amino acid sequence of “anti-CD20 arm” |
| 39 | Nucleotide sequence encoding “anti-CD3 arm” |
| 40 | Nucleotide sequence encoding “anti-CD20 arm” |
| 41 | Sequence of linker (“hinge sequence”) |
| 42 | Human IgG4 Fc region |

### EXAMPLES

The present invention, thus generally described, will be understood more readily by reference to the following Examples, which are provided by way of illustration and are, not intended to be limiting of the instant invention. The Examples are not intended to represent that the experiments below are all or the only experiments performed.

### Example 1

### Preparation of Materials and Benchmark Antibodies

### 1. Preparation of Materials

Information on the commercially available materials used in the examples are provided in Table 1.

TABLE 1

| | | Catalog |
|---|---|---|
| | | Number |
| Materials | Vendor | (Cat.) |
| Expi293 ™ Expression System Kit | Thermo Fisher | A14635 |
| | scientific | |
| Expi293 | Invitrogen | A14527 |
| ExpiFectamine293 Transfection Kit | Invitrogen | A14524 |
| Expi293 Expression Medium | Invitrogen | A1435101 |
| Protein A column | GE Healthcare | 17543802 |
| SEC column | GE Healthcare | 28990944 |
| CD4⁺T Cell Isolation Kit (Human) | Stemcell | 19052 |
| CD8⁺ T cell Isolation Kit (Human) | Stemcell | 19053 |
| Human B Cell Enrichment Kit | Stemcell | 19054 |
| Calcein-AM | Invitrogen | C3099 |
| CellTracker ™ FarRed | Invitrogen | C34572 |
| Propidium Iodide (PI) | Invitrogen | P3566 |
| PE conjugated Goat anti-human | Jackson | 109-115-098 |
| IgG Fc antibody | | |
| FITC labeled ami-human CD4; | BD Pharmingen | 550628 |
| PerCP-Cy5.5 labeled anti-human | BD Pharmingen | 560662 |
| CD8 | | |
| APC labeled anti-human CD25 | BD Pharmingen | 555434 |
| Jurkat cell line | ATCC | TIB-152 |
| Raji cell line | ATCC | CCL-86 |
| NAMALWA cell line | ATCC | CRL-1432 |
| Ramos cell line | ATCC | CRL-1596 |

### 2. Production of Benchmark Antibodies

Two benchmark antibodies BMK1 and BMK4 were applied in the examples as reference antibodies.

Anti-human CD20 benchmark antibody BMK1 (Rituximab) was generated based on the sequences of clone C2B8 from US Patent Application US 20140004037 A1. Anti-CD3×CD20 reference bispecific antibody BMK4 (REGN1979) gene was synthesized according to the sequences in patent WO 2017112762A1. The BMK antibodies were expressed from Expi293 cells and then purified using Protein A chromatography.

### Example 2

### Generation of the Bispecific Antibody T3U3-E4-1.uIgG4.SP

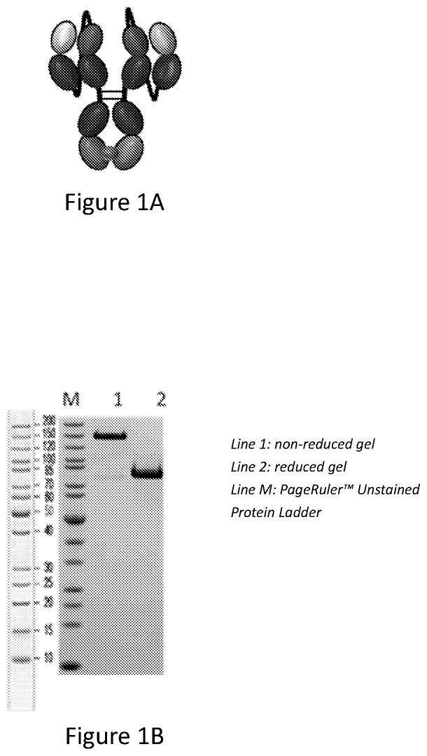

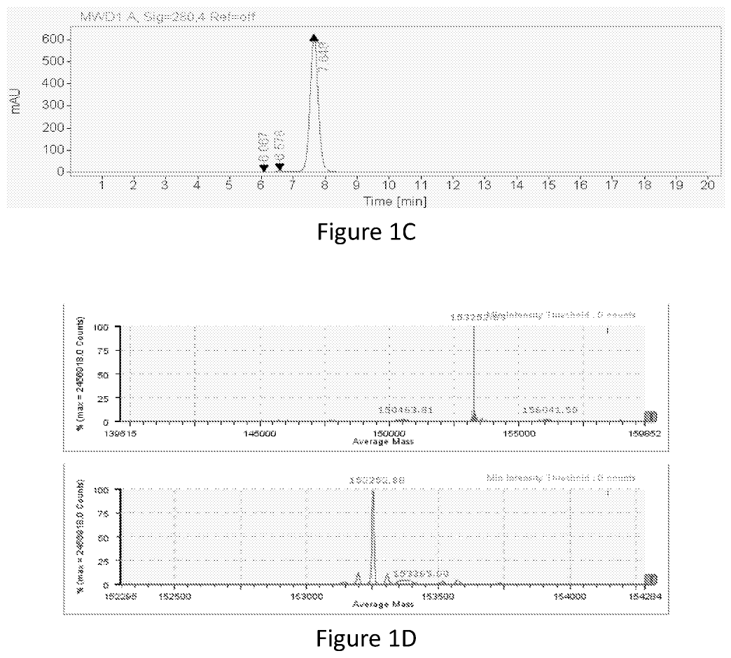

The bispecific antibody T3U3-E4-1.uIgG4.SP was produced as full-length human IgG4 in a knobs-into-holes format based on the methods described in S. Atwell, J. B. Ridgway, J. A. Wells, P. Carter, Stable heterodimers from remodeling the domain interface of a homodimer using a phage display library. J. Mol. Biol. 270, 26-35 (1997) and C. Spiess, M. Merchant, A, Huang, et al. D. G. Yansura, J. M. Scheer, Bispecific antibodies with natural architecture produced by co-culture of bacteria expressing two distinct half-antibodies. Nat, Biotechnol, 31, 753-758 (2013). The schematic diagram of the bispecific antibody T3U3-E4-1.uIgG4.SP is shown in FIG. 1A.

In the bispecilic antibody T3U3-E4-1.uIgG4.SP, the “anti-CD3 arm” (SEQ ID NO: 37) and the “anti-CD20 arm” (SEQ ID NO: 38) each is linked by a hinge sequence (SEQ ID NO: 41) to human IgG4 Fc region sequence (SEQ ID NO: 42), wherein the human IgG4 contains a S228P mutation which is present in the hinge sequence (SEQ ID NO: 41), The human IgG4 containing S228P mutation is named as “human IgG4 (S228P)” hereinafter. The anti-CD3 arm in the bispecific antibody was generated by hybridoma technology from mice immunized with human CD3ε and CD3δ extracellular domain (ECD) proteins. The preparation of the CD3 arm was also described in PCT application PCT/CN2017/102622.

The anti-CD20 arm in the bispecific antibody was based on the sequence of clone 2F2 (Ofatumumab) from PCT publication WO 2010083365A1. Both anti-CD20 and anti-CD3 arms were constructed in single chain Fab format (scFab) of VL-CL-(G₄S)₁₂-VH-CH1, which were then linked by a hinge sequence (SEQ ID NO: 41) to the constant regions of human IgG4 (S228P) CH2 and CH3. The anti-CD20 arm was assembled with anti-CD3 arm using knobs-into-holes as described in the literature [S. Atwell, J. B. Ridgway, J. A. Wells, P. Carter, Stable heterodimers from remodeling the domain interface of a homodimer using a phage display library. J. Mol. Biol. 270, 26-35 (1997) and C. Spiess, M. Merchant, A. Huang, et al. D. G. Yansura, J. M. Scheer, Bispecific antibodies with natural architecture produced by co-culture of bacteria expressing two distinct half-antibodies. Nat. Biotechnol. 31, 753-758 (2013)]. The amino acid sequence information on the CDRs and FWs of the bispecific antibody T3U3-E4-1.uIgG4.SP are listed in Table 2.

TABLE 2

| | VH of anti-CD3 arm | VL of anti-CD3 arm | VL of anti-CD20 arm | VL of anti-CD20 arm |
|---|---|---|---|---|
| FW1 | QVQLVQSGAEVK | DIVMTQSPDSLA | EVQLVESGGGLVQ | EIVLTQSPATLSL |
| | KPGSSVKVSCKAS | VSLGERATINC | PGRSLRLSCAAS | SPGERATLSC |
| | (SEQ ID NO: 21) | (SEQ ID NO: 25) | (SEQ ID NO: 29) | (SEQ ID NO: 33) |
| CDR1 | GFAFTDYYIH | KSSQSLLNSRTR | GFTFNDYAMH | RASQSVSSYLA |
| | (SEQ ID NO: 1) | KNYLA | (SEQ ID NO: 7) | (SEQ ID NO: 10) |
| | | (SEQ ID NO: 4) | | |
| FW2 | WVRQAPGQGLEW | WYQQKPGQPPK | WVRQAPGKGLEW | WYQQKPGQAPR |
| | MG | LLIY | VS | LLIY |
| | (SEQ ID NO: 22) | (SEQ ID NO: 26) | (SEQ ID NO: 30) | (SEQ ID NO: 34) |
| CDR2 | WISPGNVNTKYNE | WASTRQS | TISWNSGSIGYADS | DASNRAT |
| | NFKG | (SEQ ID NO: 5) | VKG | (SEQ ID NO: 11) |
| | (SEQ ID NO: 2) | | (SEQ ID NO: 8) | |
| FW3 | RVTITADKSTSTA | GVPDRFSGSGSG | RFTISRDNAKKSLY | GIPARFSGSGSGT |
| | YMELSSLRSEDTA | TDFTLTISSLQAE | LQMNSLRAEDTAL | DFTLTISSLEPED |
| | VYYCAR | DVAVYYC | YYCAK | FAVYYC |
| | (SEQ ID NO: 23) | (SEQ ID NO: 27) | (SEQ ID NO: 31) | (SEQ ID NO: 35) |
| CDR3 | DGYSLYYFDY | TQSHTLRT | DIQYGNYYYGMDV | QQRSNWPIT |
| | (SEQ ID NO: 3) | (SEQ ID NO: 6) | (SEQ ID NO: 9) | (SEQ ID NO: 12) |
| FW4 | WGQGTLVTVSS | FGGGTKVEIK | WGQGTTVTVSS | FGQGTRLEIK |
| | (SEQ ID NO: 24) | (SEQ ID NO: 28) | (SEQ ID NO: 32) | (SEQ ID NO: 36) |

The genes of two arms (SEQ ID NO: 40 for anti-CD20 arm; and SEQ ID NO: 39 for anti-CD3 arm) were respectively cloned into a modified pcDNA3,3 expression vector, and co-transfected into Expi293 (Invitrogen-A14527) by using ExpiFectamine293 Transfection Kit (Invitrogen-A14524). The cells were cultured in Expi293 Expression Medium (Invitrogen-A1435101) on an orbital shaker platform rotating at 135 rpm in a 37° C. incubator containing a humidified atmosphere with 8% CO₂. The culture supernatant was harvested for protein purification using Protein A column (GE Healthcare, 17543802) and then SEC column (GE Healthcare, 28990944). The protein concentration was measured by UV-Vis spectrophotometer (NanoDrop 2000, Thermo Scientific). The protein purity was estimated by SDS-PAGE (FIG. 1B) and analytic HPLC-SEC (FIG. 1C) and confirmed by MS analysis (FIG. 1D).

Results:

The purity of the bispecific antibody T3U3-E4-1.uIgG4.SP was confirmed by analytical size exclusion chromatography (SEC) and SDS-PAGE characterization. On non-reduced SDS-PAGE, the purified bispecific antibody T3U3-E4-1.uIgG4.SP was visualized as single band with corresponding MW 150 KD. On reduced SDS-PAGE, the bispecific antibody T3U3-E4-1.uIgG4.SP was visualized as 75 KD band (FIG. 1B). In analytic HPLC-SEC, the bispecific antibody T3U3-E4-1.uIgG4.SP showed a single peak with the purity above 98% (FIG. 1C). In addition, no homodimers were detected by mass spectrometry for the bispecific antibody T3U3-E4-1.uIgG4.SP (FIG. 1D).

Table 3 provides a summary on the protein yield and purity of the bispecific antibody T3U3-E4-1.uIgG4.SP in transient production.

TABLE 3

| | | Yield | purity by | Yield | purity by |
|---|---|---|---|---|---|
| | | (mg/L) | SEC-HPLC | (mg/L) | SEC-HPLC |
| Protein Name | PI | Alter Protein A purification | | After SEC purification | |
| T3U3.E4-1.uIgG4.SP | 8.0 | 217 | 81% | 128 | 98.19% |

It can be seen from FIGS. 1B, 1C and 1D as well as Table 3 that, the bispecific antibody T3U3-E4-1.uIgG4.SP were produced in a high purity (higher than 98% by SEC-HPLC) without detectable level of homodimers and aggregations.

### Examples 3

### Antibody Characterization—In Vitro Characterization

### 3.1. Cell Lines and Primary Cell Isolation

The following cell lines cultured in complete media (RPMI1640 supplemented with 10% FBS, 100 U/ml penicillin, 100 μg/ml streptomycin) were used: Jurkat (CD3⁺/CD20⁻ Raji, Ramos, Namalwa (CD20⁺/CD3⁻ cells).

Human or Cynomolgus monkey peripheral blood mononuclear cells (PBMC) were freshly isolated by Ficoll-Paque PLUS (GE Healthcare 17-1440-03) density centrifugation from heparinized venous blood from healthy donors. The primary human B cells were isolated from fresh human PBMC by EasySep kit (Stemcell-#19054), the purified CD8⁺ T cells by EasySep kit (Stemcell-19053) and purified CD4⁺ T cells by EasySep kit (Stemcell-19052).

3.2. Binding of the Bispecific Antibody T3U3-E4-1.uIgG4.SP to Target Cells

The binding of the bispecific antibody T3U3-E4-1.uIgG4.SP to target cells was determined by flow cytometry. Briefly, 1×10⁵/well of target cells (CD3⁺/CD20⁻ cells or CD20⁺/CD3⁻ cells) were incubated with serial dilutions of the bispecific. antibody T3U3-E4-1.uIgG4.SP or human IgG4 isotype control antibody at 4° C. for 60 minutes. After incubation, cells were washed twice with cold 1% BSA/1×PBS and then PE conjugated goat anti-human IgG Fc antibody (Jackson-109-115-098) was added and incubated for 30 minutes at 4° C. After washing twice, the geometric mean fluorescence (MFI) of stained cells was measured using a FACS Canto II cytometer (BD Biosciences). The EC50 values of cellbinding were determined using GraphPad Prism 5 software (GraphPad Software, La Jolla, Calif.) with values calculated using a four-parameter non-linear regression analysis.

For testing the simultaneous binding of bispecific antibody to CD3 and CD20 expressing cells. 1×10⁶/ml Raji cells and 1×10⁶/ml Jurkat cells were labeled with 50 nM Calcein-AM (Invitrogen-C3099) and 20 nM FarRed (Invitrogen-C34572), respectively. After washing with cold 1% BSA/1PBS, the labelled Raji and Jurkat cells were resuspended and mixed to a final concentration of 1×10⁶/ml at the ratio of 1:1. 1×10⁵/well of the mixed cells were plated and 20 nM of testing bispecific antibody was then added. After incubation at 4° C. for 60 minutes, the percentage of calcein-AM and FarRed double events was analyzed by FACS.

Results:

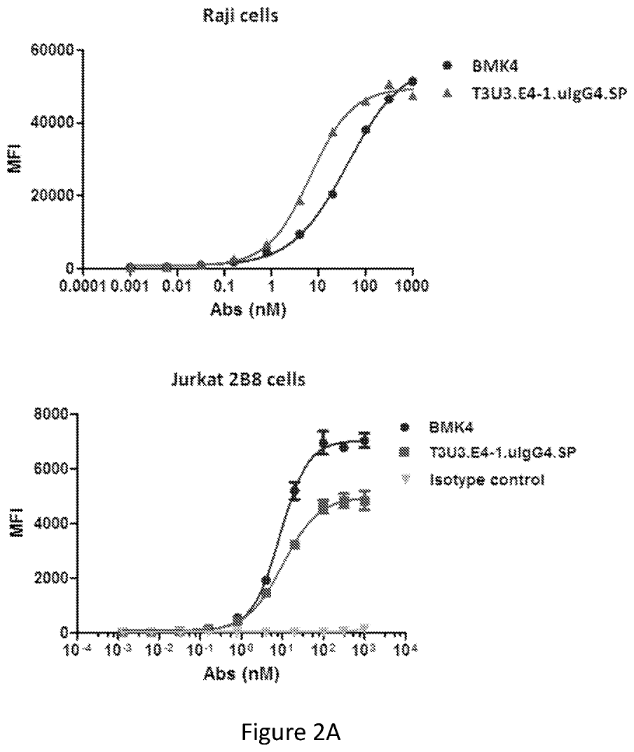

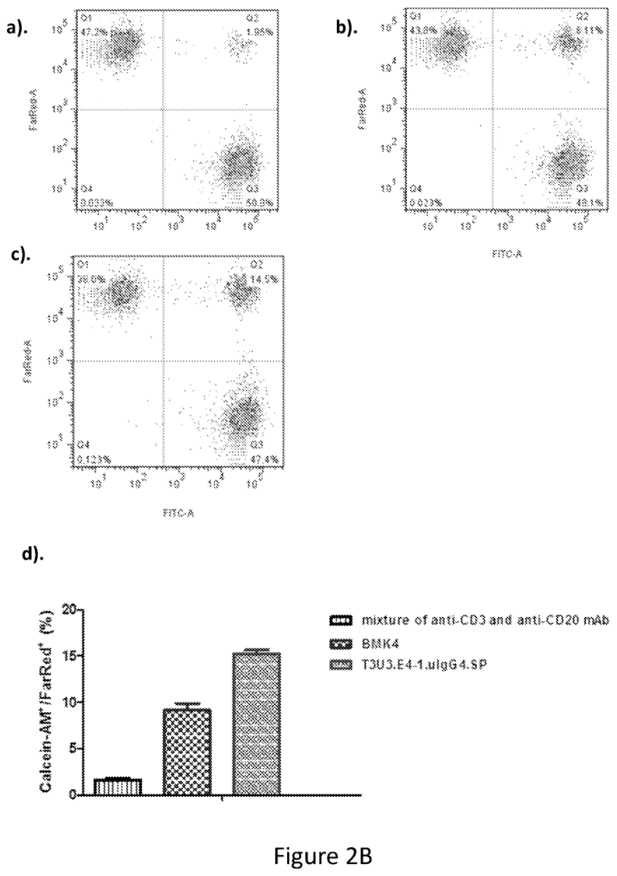

The binding of the bispecific antibody T3U3-E4-1.uIgG4.SP to cell surface CD3 and CD20 were measured by FACS with Jurkat cells and Raji cells, respectively (FIG. 2A). The binding EC50 of T3U3-E4-1.uIgG4.SP Raji cells was better than that of BMK4 and the binding EC50 to Jurkat cells was comparable to that of the BMK4 (Table 4).

TABLE 4
FACS binding EC50 of the bispecific antibody
T3U3.E4-1.uIgG4.SP to cell surface targets.

| | Raji cell (CD20) | Jurkat cells (CD3) |
|---|---|---|
| Antibody | EC50 (nM) | EC50 (nM) |
| BMK4 (REGN1979) | 41.11 | 8.80 |
| T3U3.E4-1.uIgG4.SP | 6.63 | 10.37 |

The simultaneous binding to Raji and Jurket cells of bispecific antibody was evaluated by FACS. The Calcein-AM labelled Raji cells and FarRed labelled Jurkat cells were mixed at the ratio of 1:1 and then incubated with 20 nM testing bispecific antibody as indicated in FIG. 2B. The Calcein-AM and FarRed double positive events representing the bispecific antibody bridged Raji and Jurkat cells were shown in the upper right part of the FACS scatter diagram (FIG. 2B, a-c). The bar graph of % double positive events indicated that The bispecific antibody T3U3-E4-1.uIgG4.SP was better than BMK4 in the simultaneous dual target binding (FIG. 2B, d).

### 3.3. Affinity of the Bispecific Antibody Measured by FACS

Binding affinity of bispecific antibody to cell surface CD20 or CD3 was determined by flow cytometry with Ramos and Jurkat cell lines, respectively. The cells were washed with PBS and resuspended in 1% BSA/1×PBS at 1×10⁶ cell/ml. 50 μl cell suspension was added to each well of 96-well U-plate (Corning, USA). The plates were then centrifuged at 1500 rpm for 4 min and supernatants were discarded. The serial dilutions of bispecific antibody of 100 μl/well in 1% BSA/1×PBS were added to the plates. After incubation at 4° C. for 1 hour, the cells were centrifuged at 1500 rpm for 4 min. Each cell pellet was resuspended with 100 μl/well FITC labelled goat anti-human IgG Fc (Jackson, Cat No.109-095-098, Lot No.114130). After incubation at 4° C. for 30 min, the cells were washed once with 1% BSA/1×PBS and re-suspended in 100 μ/l/well 1% BSA/1×PBS for flow cytometry analysis (BD, Cantoll, USA). The Bₘₐₓ and the K_{D} were assessed by FACS analysis and calculated by linear regression curve in GraphPad Prism 5. The K_{D} value=free IgG *(Bₘₐₓ−bound IgG)/bound IgG. The bound IgG and free IgG were calculated based on FITC Beads Quantity Equation (Quantum™ MESF Kits, Bangs Laboratories)

Results:

Table 5 shows the affinity of bispecific antibody to cell surface targets measured by FACS. The KD values of T3U3.E4-1.uIgG4.SP to CD20 on Ramos cells and to CD3 on Jurkat cells were better than that of BMK4

TABLE 5
Affinity of bispecifie antibody to cell surface targets measured by FACS

| | | BMK4 | | BMK4 |
|---|---|---|---|---|
| Sample | T3U3.E4-1.uIgG4.SP | (REGN1979) | T3U3.E4-1.uIgG4.SP | (REGN1979) |
| Cells | Ramos | | Jurkat | |
| Bₘₐₓ | 8.5 × 10⁻¹⁰ | 6.8 × 10⁻¹⁰ | 1.1 × 10⁻¹⁰ | 1.1 × 10⁻¹⁰ |
| K_{D} | 2.7 × 10⁻⁸ | 6.7 × 10⁻⁸ | 3.5 × 10⁻⁹ | 3.0 × 10⁻⁸ |
| r² | 1.00 | 0.99 | 1.00 | 0.99 |

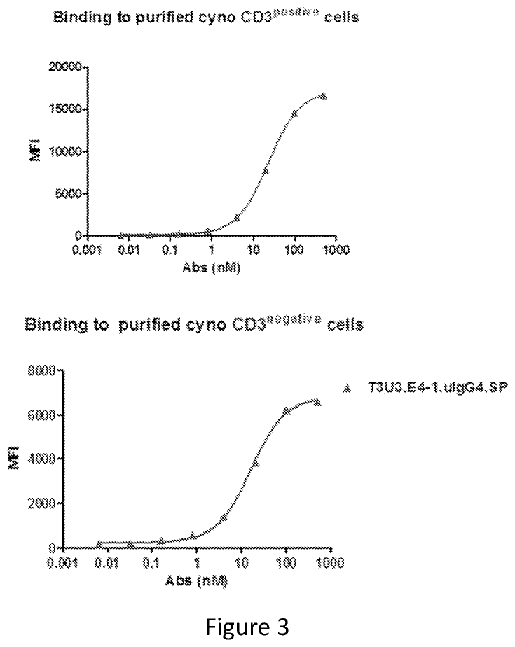

The binding of the bispecific antibody T3U3-E4-1.uIgG4.SP to cynomolgus monkey targets were tested by FACS with PBMC gating with CD3 expression (CD3 negative cells and positive cells in PBMC). The result show that the bispecific antibody T3U3-E4-1.uIgG4.SP bound to cynomolgus targets on cell surface in a dose response manner (FIG. 3).

### 3.4. T Cell Activation Assay

T activation by bispecific antibody was determined by flow cytometry measuring the percentage of CD25 expressing effector cells. Freshly isolated purified CD4⁺ T cells and CD8⁺ T cells were examined as effector cells, respectively. Briefly, 5×10⁴ effector cells were plated in 110 μl/well of complete media containing serial dilution of bispecific antibody or hIgG4 isotype control antibody, in the presence or absence of 1×10⁴ Raji cells/well, for 24 hours at 37° . After incubation, the cells were washed twice with 1% BSA/1×DPBS and then stained with anti-human Ab panel (FITC labeled anti-human. CD4 (BD Pharmingen-550628): PerCP-Cy5.5 labeled anti-human CD8 (BD Pharmingen-560662) and APC labeled anti-human CD25 (BD Pharmingen-555434)) at 4° C. for 30 minutes. T cell activation evaluated by CD25 expression was analyzed by FACS. EC50 of T-cell activation was determined by using Prism four-parameter non-linear regression analysis.

Results:

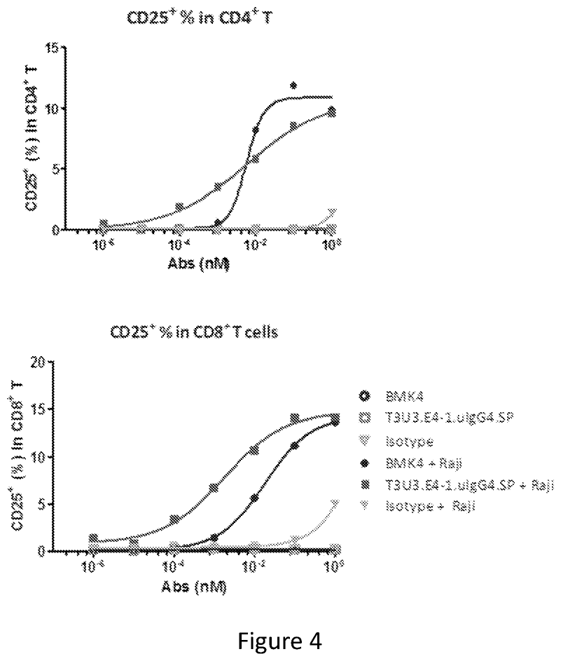

The activations of CD4⁺ and CD8⁺ T cells were measured for CD25 expression by FACS. The T cell activation mediated by the bispecific antibody could only be observed in the presence of Raji cells (FIG. 4. solid lines and symbols). In contrast, no T cell activation could be induced in the absence of Raji cells (FIG. 4. dotted lines and open symbols). The EC50 values of T cell activation by the bispecific antibody T3U3-E4-1.uIgG4.SP and BMK4 were shown in Table 6. These results indicated that. T3U3-E4-1.uIgG4.SP was more potent than BMK4 in mediating T cell activation, which was strictly dependent on the presence of target Raji cells.

TABLE 6
EC50 of T cell activation mediated by the bispecific
antibody in the presence of Raji cells

EC50 (pM)

| Abs | CD4⁺ cells | CD8⁺ cells |
|---|---|---|
| BMK4 (REGN1979) | 6.0 pM | 18.3 pM |
| T3U3.E4-1.uIgG4.SP | 6.0 pM | 1.8 pM |

### 3.5. In VitroCytotoxicity Assays

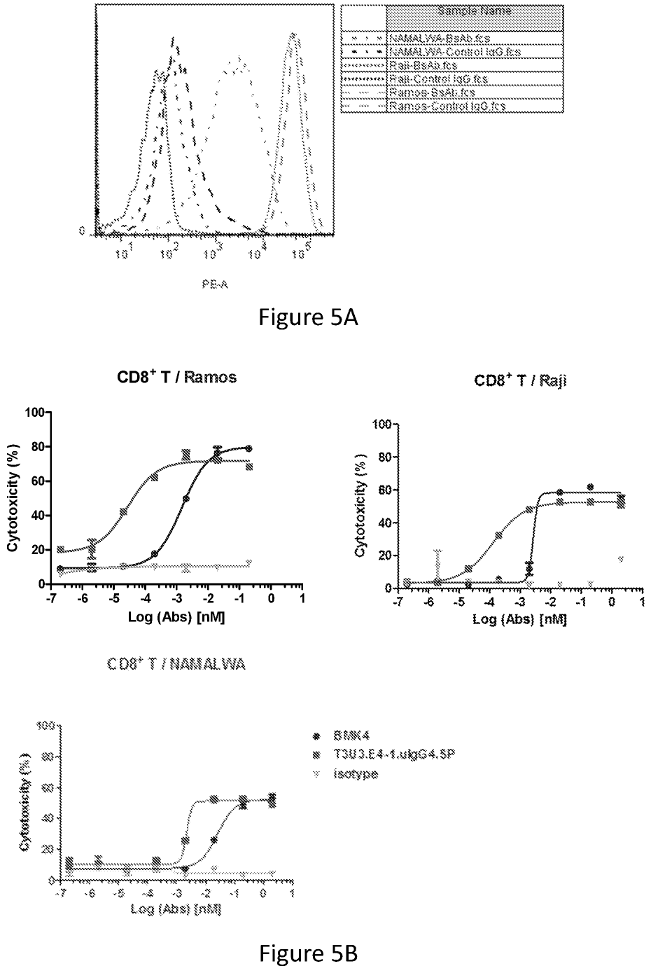

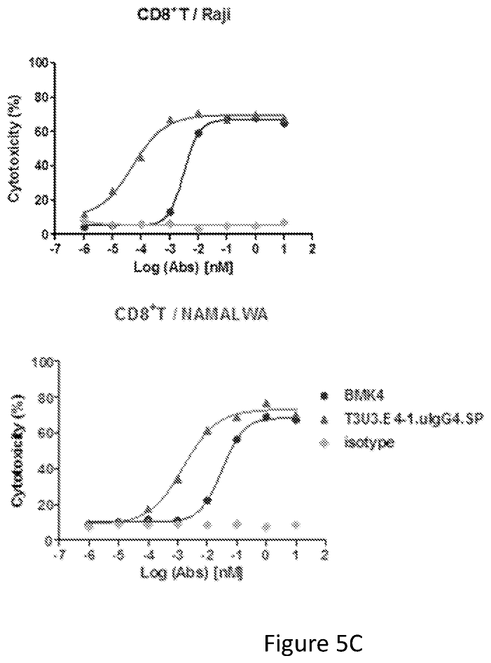

The efficacy of bispecific antibody to mediate tumor cell lysis by CD8⁺ T lymphocytes was determined by calcein release assay (FIG. 5B) and FACS based cytotoxicity assay (FIG. 5C).

For calcein release assay, the freshly isolated CD8⁺ T cells or PBMC were cultured overnight in complete media containing 50 IU/ml recombinant human IL-2. On the next day, 1×10⁶ cells/ml Raji, Ramos and NAMALWA cells were labeled with 1 μM Calcein-AM (Invitrogen-C3099) for 30 minutes at 37° C. in assay buffer (Phenol red free RPMI 1640 culture medium+10% FBS), respectively. 5×10³ cells/well of Calcein-labeled target cells were Plated in 110 μl/well of complete media containing activated effector CD8⁺ T cells (effector CD8³⁰ T cell/target cell ratio=5:1) and serial dilution of testing Abs. After incubation at 37° C. for 2 hours, the plates were centrifuged and supernatants were transferred to a translucent black clear bottom plate (Greiner-655090) for fluorescence analysis by EnVision (PerkinElmer). The percent cytotoxicity was calculated using the equation as: % cytotoxicity=(F_{S}−F_{SR})/(F_{MR}−F_{SR})*100%. Where F_{S} is calcein release from the test well; F_{SR} is spontaneous calcein release; F_{MR} is maximal calcein release from the cells lysed by Triton-X100. Results are expressed as the % specific lysis (mean±SD) from duplicate or triplicate wells.

For FACS based cytotoxicity assay, the freshly isolated human CD8⁺ T cells were cultured overnight in complete media containing 50 IU/ml recombinant human IL-2. At the following day, Raji and NAMALWA (1×10⁶ cells/ml) were respectively labeled with 20 nM. Far-Red (Invitrogen-C341572) in DPBS for 30 minutes at 37 and then washed twice with assay buffer (Phenol red free RPM1 1640 culture medium+10% FBS), Me Far-Red-labeled target cells (2×10⁴/well) were plated in 110 μl/well complete media containing effector CD8⁺ T cells (effector/target cell ratio 5:1) and serial dilutions of test Abs. After incubation at 37° C. for 4 hours, Propidium Iodide (PI) (Invitrogen-P3566) was added and incubated for 15 minutes at room temperature before analysis by flow cytometry. Percent cytotoxicity was calculated using the equation as: Cytotoxicity %=Far Red⁺PI⁺/(Far Red⁺PI⁺+Far Red⁺PI⁺)*100. The EC₅₀ values of in vitro cytotoxicity were determined using Prism four-parameter non-linear regression analysis.

Results:

The cytotoxicity of human B lymphoma cell lines mediated by bispecific antibody was tested by calcein release cytotoxicity assay (FIG. 5B) and FACS based cynitoxicity assay (FIG. 5C). The three human B lymphoma cell lines expressed different levels of cell surface CD20, detected with T3U3-E4-1.uIgG4.SP and PE conjugated goat anti-human IgG Fc antibody (Jackson-109-115-098), measured by FACS. The CD20 expression level of NAMALWA cells was lower than that of Romas and Raji cells (FIG. 5A). The FIGS. 5C and 5B showed that the cytotoxicity mediated by the bispecific antibody T3U3-E4-1.uIgG4.SP was in a dose-dependent manner and the cytotoxicity efficacies were proportionally increased with cell surface CD20 expression levels. Specifically, as shown in FIG. 5B, the bispecific antibody T3U3-E4-1.uIgG4.SP is more potent than BMK4 at mediating cytotoxicity of three different B lymphoma cell lines in two hour calcein release assays, measured by Envision. Further, as shown in FIG. 5C, the bispecific antibody T3U3-E4-1.uIgG4.SP is more potent than BMK4 at mediating cytotoxicity of two different B lymphoma cell lines in FACS based cytotoxicity assays.

The cytotoxicity EC50 values by the bispecific antibody T3U3-E4-1.uIgG4.SP and by BMK4 were summarized in Table 7 and Table 8. The results indicated that the bispecific antibody T3U3-E4-1.uIgG4.SP was more potent than BMK4 in in vitro cytotoxicity assays.

TABLE 7
EC50 of bispecific antibody mediated cytotoxicity
of three B lymphoma cell lines in two hour calcein
release assays measured by Envision.

| | Raji | Ramos | NAMALWA |
|---|---|---|---|
| Abs | EC50 (pM) | EC50 (pM) | EC50 (pM) |
| BMK4 (REGN1979) | 2.69 | 1.49 | 26.01 |
| T3U3.E4-1.uIgG4.SP | 0.13 | 0.02 | 2.23 |

TABLE 8
EC50 of bispecific antibody mediated cytotoxicity of two B lymphoma
cell lined in 4 hours FACS based cytotoxicity assays.

| | Raji | NAMALWA |
|---|---|---|
| Abs | EC50 (pM) | EC50 (pM) |
| BMK4 (REGN1979) | 3.12 | 31.83 |
| T3U3.E4-1.uIgG4.SP | 0.05 | 1.64 |

### 3.6. DSF Assay and Thermal Stability Test

A DSF assay was performed using 7500 Fast Real-Time PCR system (Applied Biosystems). Briefly, 19 μL of antibody solution was mixed with 1 μL of 62.5×SYPRO Orange solution (Invitrogen) and added to a 96 well plate (Biosystems). The plate was heated from 26° C. to 95° C. at a rate of 2° C./min, and the resulting fluorescence data were collected. The negative derivatives of the fluorescence changes with respect to different temperatures were calculated, and the maximal value was defined as nicking temperature Tₕ. If a protein has multiple unfolding transitions, the first two Tₕ were reported, named as Tₕ₁ and Tₕ₂. Tₕ₁ is always interpreted as the formal melting temperature Tₘ to facilitate comparisons between different proteins. Data collection and Tₕ calculation were conducted automatically by its operation software. Once the plot of negative derivatives of different temperatures was reported by the software, the point in the plot where the curve starts to decrease from a pre-transition baseline could be roughly estimated as the onset temperature Tₒₙ.

Results:

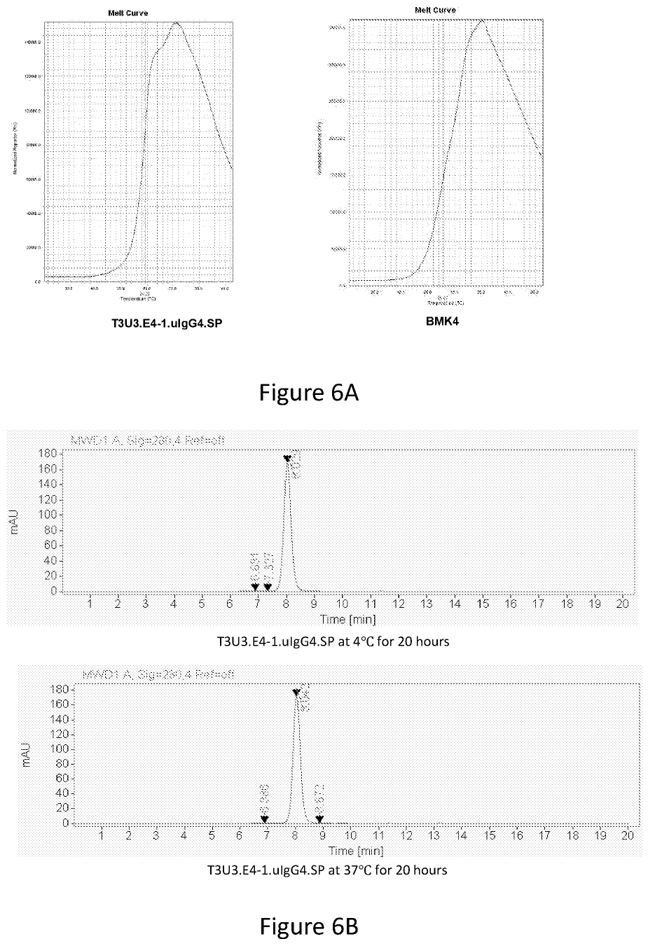

The thermal stability of bispecific antibody was test by DSF assay, where the Tₒₙ, Tₕ₁ and Tₕ₂ of T3U3-E4-1.uIgG4.SP and BMK4 were in normal range and the DSF profiles were normal (FIG. 6A). The Tₘ, interpreted as Tₕ₁, of T3U3-E4-1.uIgG4.SP was 64.4° C., which was better than that of BMK4 (57.6° C.) (Table 9).

TABLE 9
Thermal stability of the bispecific antibody.

| Abs | PI | Tₒₙ (° C.) | Tₕ₁ (° C.) | Tₕ₂ (° C.) |
|---|---|---|---|---|
| T3U3.E4-1.uIgG4.SP | 8.00 | 47 | 64.4 | 73.7 |
| BMK4 (REGN1979) | 7.65 | 45 | 57.6 | 72.6 |

The thermal stability of the Bispecific antibody was further conducted by incubating the test antibody aliquots at 4° C. and 37° C. for 20 hours. After the incubation, the test antibody aliquots were removed and subjected to SEC-HPLC analysis to detect main peak purity (monomer content).

Results:

After incubation, the stability of the bispecific antibody T3U3-E4-1.uIgG4.SP was tested by analytic HPLC-SEC, which showed high purity and free of polymers and degradations (FIG. 6B).

These results indicated that the bispecific antibody T3U3-E4-1.uIgG4.SP was stable in thermal stability tests.

### 3.7. Serum Stability Test

Freshly collected human blood was incubated in polystyrene tubes without anticoagulant for 30 minutes at room temperature. The serum was collected after centrifugation the blood at 4000 rpm for 10 minutes.. The antibodies were gently mixed with serum to ensure the serum content >95%. The mixed aliquots were incubated at 37° C. for 0 day, 1 day, 4 days, 7 days and 14 days, respectively. At the indicated time point the samples were quickly-frozen in liquid nitrogen and stored at −80° C. until analysis. The cell bindings of the serial dilution of the samples to Raji and Jurkat cells were analyzed by FACS, respectively. Prism four-parameter non-linear regression was used to analyze cell binding.

Results:

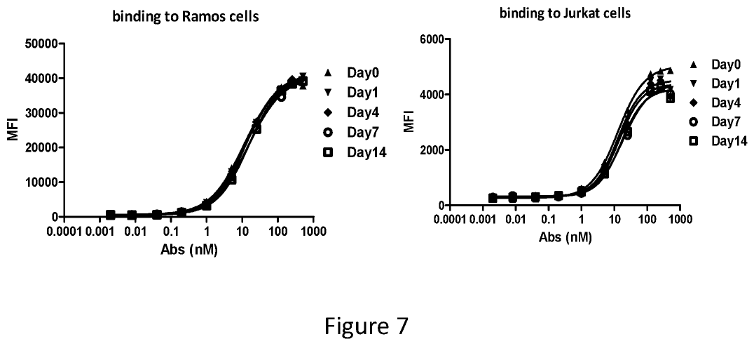

After each incubation time period, the binding activity of bispecific antibody to target cells were compared to that of freshly thawed bispecific antibody (day 0). As shows in FIG. 7, the binding of the bispecific antibody T3U3-E4-1.uIgG4.SP to both Ramos and Jurkat cells were kept normal after incubation in human serum. These results suggested that T3U3-E4-1.uIgG4.SP was stable in human serum for at least 14 days.

### 3.8. Alkaline Stress Test

The test antibody was buffer exchanged into an alkaline buffer (20 mM Tris, 150 mM NaCl, pH 8.5) using microcentrifuge desalting column t 7K MWCO, Thermo Fisher, Cat. No.: Pierce-89889). Concentration of antibody was detected by UV-Vis spectrophotometer (NanoDrop 2000, Thermo Scientific). The antibody was then incubated at 37° C. for 5 days. Binding affinity of the stressed or non-stressed antibody to target cells were tested for evaluation of Ab stability in stress test.

Results:

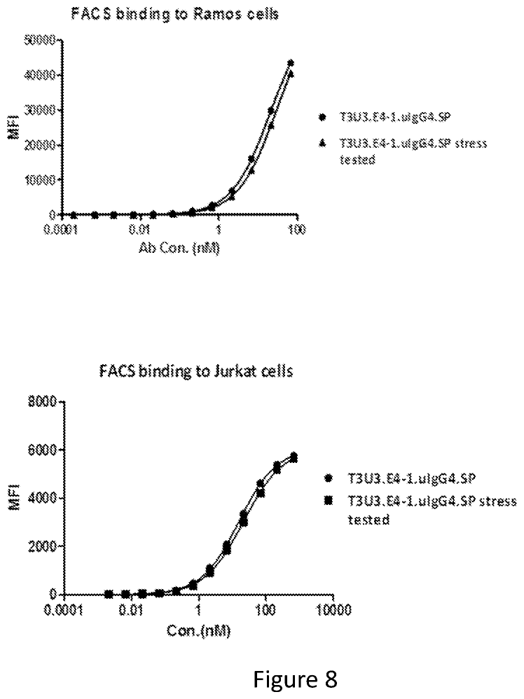

The stability of the bispecific antibody T3U3-E4-1.uIgG4.SP in alkaline stress test was tested by incubation of the bispecific antibody in the alkaline buffer at 37° C. for 5 days. After incubation, the bindings of the stressed bispecific antibody to target cells were compared to that of freshly thawed bispecific antibody. As shown in FIG. 8, the binding curves of the stressed bispecific antibody T3U3-E4-1.uIgG4.SP to both Ramos and Jurkat cells were normal as compared to the untreated antibody, suggesting the bispecific antibody was stable in alkaline stress test.

3.9. Non-Specific Binding

The antibody non-specific bindings were test by ELISA and FACS to multiple irrelevant proteins and cells, respectively. Non-specific binding ELISA was performed in 96-well high binding plates (Nunc-Immuno Plate, Thermo Scientific). The plate was coated with various antigens at 2 μg/mL overnight at 4° C. After blocking with 2% BSA-PBS, 10 μg/ml test antibodies were added to the plate and incubated for 2 hours. The plates were subsequently incubated with the secondary antibody goat anti human IgG Fc-HRP (Bethyl) for additional 1 hour. The HRP signal was detected by adding TMB peroxidase substrate and the reaction was stopped after 12 minute using 2M HCl. The absorbance at 450 nm was read using a microplate reader (Molecular Device). All incubation steps were performed at room temperature. The plate was washed with PBST (0.05% Tween20-PBS) between steps. For non-specific binding FACS various cell lines were used. Briefly, the viable cells were centrifuged at 1500 rpm for 4 min and then re-suspended in an appropriate volume of 1% BSA/1×PBS to the concentration of 1×10⁶ cell/ml. 100 μl cell suspension was added into each well of 96-well U-plate. After centrifugation, the cells were re-suspended with 100 μl/well diluted test antibodies at 10 μg/ml in 1% BSA/1×PBS. After incubation at 4° C. for 1 hour, the cells were washed twice with 1% BSA/1×PBS then incubated with 5 μg/ml goat anti-human IgG Fc-PE (Jackson, 109-115-098 & 126973) at 4° C. for 30 min. After two time of washing, the cells were re-suspended in 100% BSA/1×PBS and kept at 4° C. in the dark until FACS analysis (BD Canto II).

Results:

No non-specific binding was observed for the bispecific antibody T3U3-E4-1.uIgG4.SP in the tests, as shown in Table 10A and 10B.

TABLE 10A
Non-specific binding test of the bispecific antibody T3U3.E4-1.uIgG4.SP by ELISA.

| | Factor | | | | | | | | | | No |
|---|---|---|---|---|---|---|---|---|---|---|---|
| Ab (10 ug/ml) | Eight | FGFR.his | PD-1.his | CTLA4.his | CD22.his | VEGF.his | CD3.his | HER3.his | OX40.his | 4-1BB.his | coating |
| T3U3.E4- | 0.16 | 0.22 | 0.14 | 0.18 | 0.12 | 0.15 | 3.56* | 0.14 | 0.10 | 0.11 | 0.12 |
| 1.uIgG4.SP | | | | | | | | | | | |
| Human IgG4K | 0.39 | 0.19 | 3.87* | 0.13 | 0.11 | 0.19 | 0.12 | 0.10 | 0.08 | 0.10 | 0.10 |
| control | | | | | | | | | | | |
| Human IgG4L | 0.11 | 0.19 | 0.11 | 0.10 | 0.11 | 0.13 | 0.11 | 0.11 | 0.09 | 0.10 | 0.11 |
| control | | | | | | | | | | | |
| 2ₒₙ Ab (Goat | 0.08 | 0.18 | 0.09 | 0.08 | 0.09 | 0.10 | 0.10 | 0.08 | 0.06 | 0.08 | 0.08 |
| anti-human IgG | | | | | | | | | | | |
| Fc-HRP) | | | | | | | | | | | |

*specific binding

TABLE 10B
Non-specific binding test of the bispecific antibody T3U3.E4-1.uIgG4.SP by FACS.

| | FACS MFI | | | | | | | | |
|---|---|---|---|---|---|---|---|---|---|
| Ab | | | MDA- | | | | | | |
| (10 ug/ml) | Ramos | Raji | MB453 | BT474 | Jurkat | Hut78 | A431 | A204 | CaLu-6 |
| Blank | 30 | 29 | 33 | 28 | 22 | 29 | 25 | 24 | 25 |
| 2ₒₙ AB (Goat Anti- | 65 | 67 | 34 | 24 | 21 | 28 | 25 | 24 | 25 |
| human IgG Fc -PE) | | | | | | | | | |
| T3U3.E4-1uIgG4.SP | 38500* | 43400* | 42 | 35 | 2556* | 4069* | 110 | 24 | 109 |
| Human IgG4K control | 68 | 92 | 106 | 35 | 25 | 61 | 30 | 26 | 28 |
| Human IgG4L control | 63 | 72 | 45 | 30 | 22 | 38 | 27 | 24 | 28 |

| Ab | FACS MFI | | | | | | |
|---|---|---|---|---|---|---|---|
| (10 ug/ml) | A375 | HepG2 | BxPC-3 | HT29 | FaDu | 293F | CHO-K1 |
| Blank | 37 | 32 | 34 | 25 | 23 | 31 | 32 |
| 2ₒₙ AB (Goat Anti- | 34 | 33 | 35 | 24 | 23 | 31 | 30 |
| human IgG Fc -PE) | | | | | | | |
| T3U3.E4-1.uIgG4.SP | 46 | 43 | 76 | 57 | 98 | 56 | 31 |
| Human IgG4K control | 39 | 48 | 39 | 28 | 29 | 46 | 32 |
| Human IgG4L control | 35 | 36 | 35 | 27 | 25 | 34 | 32 |

*specific binding

### Example 4

### Antibody Characterization—In Vivo Characterization

### In Vivo Tumor Efficacy

The antibody in vivo anti-tumor efficacy was tested in an admixed PBMC humanized model bearing Raji tumor in NOG mice. Female NOG mice (Beijing Vital River Laboratory Animal Technology Co., LTD) of 6-8 week-old were used in the studies. The Raji tumor cells (ATCC® CCL-86™) were maintained in vitro as a monolayer culture in 1640 medium supplemented with 10% fetal bovine serum, 100 U/ml penicillin and 100 μg/ml streptomycin at 37° C. in an atmosphere of 5% CO₂ in air, the tumor cells were routinely sub-cultured twice weekly. The cells growing in an exponential growth phase were harvested and counted for tumor inoculation. Human PBMC were isolated from heparin whole blood of a single healthy donor by using Ficoll-Paque Plus per manufacturer's instructions.

For prophylactic model, Raji cells (2.0×10⁶) were co-implanted with fresh isolated PBMC (3.0×10⁶) or in vitro activated PBMC (2.0'10⁶) subcutaneously into NOG mice and Ab treatment was started at the same day. The in vitro activated PBMC were prepared by stimulating fresh PBMC with OKT3 antibody for 4 days before injection. The mice were treated with antibodies intravenously twice weekly from day 0 for 3 weeks. The group information was described in Table 11A.

For therapeutic model, each mouse was co-inoculated subcutaneously at the right upper flank with pre-mixed Raji tumor cells (2.0×10⁶) and fresh isolated PBMC (3.0×10⁶). When the average tumor volume reached approximately to 74 mm³, the animals were randomized for grouping and received the first antibody injection. For the efficacy study the mice were treated with antibodies intravenously twice weekly for 3 weeks. The group information was described in the Table 11B.

All the procedures related to animal handling, care and the treatment in the study were performed according to the guidelines approved by the Institutional Animal Care and Use Committee (IACUC) of WuXi AppTec following the guidance of the Association for Assessment and Accreditation of Laboratory Animal Care (AAALAC). For all tumor studies, mice were weighed and tumor growth was measured twice a week using calipers. Tumor volume was estimated as ½ (length×width).

TABLE 11A
Group information of prophylactic model

| Group | N^{a} | Raji Cell | PBMC | Antibody | Dose^{b} | |
|---|---|---|---|---|---|---|
| 1 | 8 | 2.0 × 10⁶ | fresh, 3 × 10⁶ | human IgG4 isotype control | 2 | mg/kg |
| 2 | 8 | 2.0 × 10⁶ | fresh, 3 × 10⁶ | BMK4 | 1.5 | mg/kg |
| 3 | 8 | 2.0 × 10⁶ | fresh, 3 × 10⁶ | T3U3.E4-1.uIgG4.SP | 1.5 | mg/kg |
| 4 | 6 | 2.0 × 10⁶ | activated, 2 × 10⁶ | human IgG4 isotype control | 1.5 | mg/kg |
| 5 | 5 | 2.0 × 10⁶ | activated, 2 × 10⁶ | T3U3.E4-1.uIgG4.SP | 1.5 | mg/kg |

TABLE 11B
Group information of therapeutic model

| Group | N^{a} | Raji Cell | Fresh PBMC | Antibody | Dose^{b} | |
|---|---|---|---|---|---|---|
| 1 | 6 | 2.0 × 10⁶ | 3 × 10⁶ | human IgG4 isotype control | 5 | mg/kg |
| 2 | 6 | 2.0 × 10⁶ | 3 × 10⁶ | BMK4 | 5 | mg/kg |
| 3 | 7 | 2.0 × 10⁶ | 3 × 10⁶ | BMK4 | 0.5 | mg/kg |
| 4 | 6 | 2.0 × 10⁶ | 3 × 10⁶ | BMK4 | 0.05 | mg/kg |
| 5 | 6 | 2.0 × 10⁶ | 3 × 10⁶ | T3U3.E4-1.uIgG4.SP | 5 | mg/kg |
| 6 | 7 | 2.0 × 10⁶ | 3 × 10⁶ | T3U3.E4-1.uIgG4.SP | 0.5 | mg/kg |
| 7 | 7 | 2.0 × 10⁶ | 3 × 10⁶ | T3U3.E4-1.uIgG4.SP | 0.05 | mg/kg |
| 8 | 6 | 2.0 × 10⁶ | 3 × 10⁶ | BMK1 | 0.5 | mg/kg |

^{a}number of animals per group;
^{b}each mouse received dose volume: 10 ml/kg.

Results:

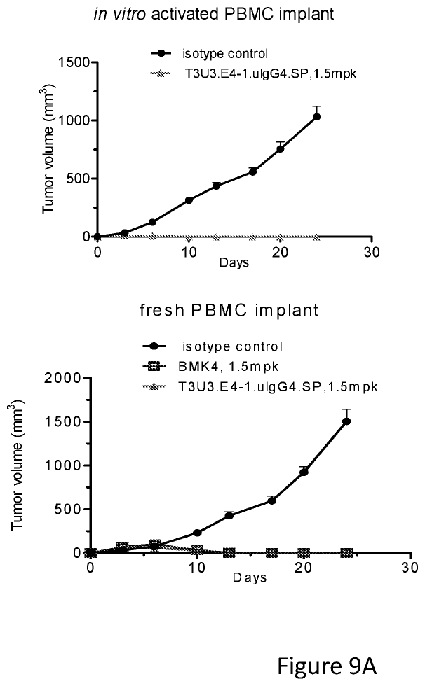

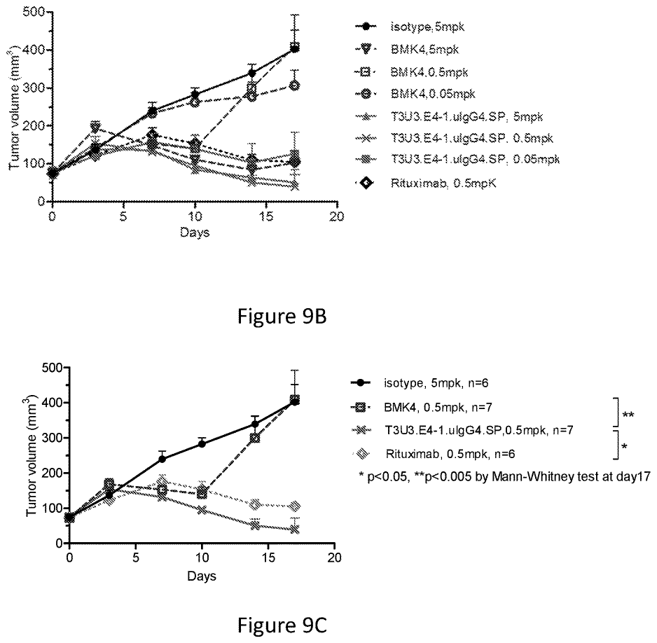

In the prophylactic model, immediate treatment with Abs at day 0 resulted in complete prevention of tumor outgrowth at all tested mice, including all of the bispecific antibody T3U3-E4-1.uIgG4.SP and BMK4 treated mice (FIG. 9A).

In therapeutic model when the Raji tumors have established, the mice were received three different doses (0.05 mg/kg or 0.5 mg/kg, or 5 mg/kg) of Ab treatments twice per week for 3 weeks. The bispecific antibody T3U3-E4-1.uIgG4.SP could induce tumor growth inhibition at all tested doses, in contrast, BMK4 only at the highest dose of 5 mg/kg. In addition, the bispecific antibody T3U3-E4-1.uIgG4.SP at 0.05 mg/kg showed equally effective as Rituximab at 0.5 mg/kg and BMK4 at 5 mg/kg for significantly inhibiting tumor growth (FIG. 9B).

Furthermore, as shown in FIG. 9C and Table 12, when comparing the Ab efficacy at the equal mole dose (=0.5 mg/kg), the Bispecific antibody T3U3-E4-1.uIgG4.SP exhibited the most potent efficacy as compared with BMK1(Rituximab) and BMK4 for inhibiting tumor growth and eradicating tumor.

TABLE 12
Comparison of Ab efficacy on inhibiting
in vivo tumor growth at equal mole dose.

| | | | | Tumor |
|---|---|---|---|---|
| Abs | Tumor volume | T/C | TGI | eradication |
| (0.5 mg/kg) | (mm³) | (%) | (%) | rate (%) |
| Isotype control | 402 | — | — | 0 |
| BMK4 (REGN1979) | 399 | 99.1 | 0.5 | 0 |
| T3U3.E4-1.uIgG4.SP | 38 | 9.5 | 111.6 | 71.4 |
| BMK1 (Rituximab) | 105 | 26.1 | 90.5 | 0 |

In summary, these results demonstrated that T3U3-E4-1.uIgG4.SP was very potent in in vivo anti-tumor activity.

Those skilled in the art will further appreciate that the present invention may be embodied in other specific forms without departing from the spirit or central attributes thereof. In that the foregoing description of the present invention discloses only exemplary embodiments thereof, it is to be understood that other variations are contemplated as being within the scope of the present invention. Accordingly, the present invention is not limited to the particular embodiments that have been described in detail herein. Rather, reference should be made to the appended claims as indicative of the scope and content of the invention.

1.-50. (canceled)

51. A bispecific antibody or the antigen-binding portion thereof, comprising a first antigen-binding site that specifically binds to CD3 and a second antigen-binding site that specifically binds to CD20,
wherein the first antigen-binding site comprises in the heavy chain variable region a CDR (complementarity determining region) 1 of SEQ ID NO: 1, a CDR2 of SEQ ID NO: 2, and a CDR3 of SEQ ID NO: 3, and in the light chain variable domain a CDR1 of SEQ ID NO: 4, a CDR2 of SEQ ID NO: 5, and a CDR3 of SEQ ID NO: 6.

52. The bispecific antibody or the antigen-binding portion thereof of claim 51, wherein the first antigen-binding site comprises in the heavy chain variable region:
(i) the amino acid sequence of SEQ ID NO: 13;
(ii) an amino acid sequence at least 85%, 90%, or 95% identical to the amino acid sequence of SEQ ID NO: 13; or
(iii) an amino acid sequence with addition, deletion and/or substitution of one or more amino acids compared with the amino acid sequence of SEQ ID NO: 13; or
wherein the first antigen-binding site comprises in the light chain variable region comprises:
(i) the amino acid sequence of SEQ ID NO: 14;
(ii) an amino acid sequence at least 85%, 90%, or 95% identical to the amino acid sequence of SEQ ID NO: 14; or
(iii) an amino acid sequence with addition, deletion and/or substitution of one or more amino acids compared with the amino acid sequence of SEQ ID NO: 14.

53. The bispecific antibody or the antigen-binding portion thereof of claim 51, wherein the second antigen-binding site comprises in the heavy chain variable region a CDR1 of SEQ ID NO: 7, a CDR2 of SEQ ID NO: 8, and a CDR3 of SEQ ID NO: 9, and in the light chain variable domain a CDR1 of SEQ ID NO: 10, a CDR2 of SEQ ID NO:11, and a CDR3 of SEQ ID NO: 12.

54. The bispecific antibody or the antigen-binding portion thereof of claim 51, wherein the second antigen-binding site comprises in the heavy chain variable region comprises:
(i) the amino acid sequence of SEQ ID NO: 15;
(ii) an amino acid sequence at least 85%, 90%, or 95% identical to the amino acid sequence of SEQ ID NO: 15; or
(iii) an amino acid sequence with addition, deletion and/or substitution of one or more amino acids compared with the amino acid sequence of SEQ ID NO: 15; or
wherein the second antigen-binding site comprises in the light chain variable region comprises:
(i) the amino acid sequence of SEQ ID NO: 16;
(ii) an amino acid sequence at least 85%, 90%, or 95% identical to the amino acid sequence of SEQ ID NO: 16; or
(iii) an amino acid sequence with addition, deletion and/or substitution of one or more amino acids compared with the amino acid sequence of SEQ ID NO: 16.

55. The bispecific antibody or the antigen-binding portion thereof of claim 51, wherein the bispecific antibody or the antigen-binding portion thereof further comprise a Fc region, preferably a human Fc region, more preferably a human IgG Fc region, most preferably a human IgG4 Fc region, wherein the IgG4 Fc region is represented by SEQ ID NO: 42.

56. The bispecific antibody or the antigen-binding portion thereof of claim 51, wherein the bispecific antibody or the antigen-binding portion thereof comprises a hinge sequence, preferably wherein the hinge sequence is represented by SEQ ID NO: 41.

57. The bispecific antibody or the antigen-binding portion thereof of claim 51, wherein the bispecific antibody or the antigen-binding portion thereof is in a knobs-into-holes format.

58. The bispecific antibody or the antigen-binding portion thereof of claim 51, wherein the bispecific antibody or the antigen-binding portion thereof is a humanized antibody.

59. The bispecific antibody or the antigen-binding portion thereof of claim 51, wherein the bispecific antibody or the antigen-binding portion thereof binds to cell surface human CD20 with a K_{D} of 1×10⁻⁷ M or less, as measured by FACS, and/or
wherein the bispecific antibody or the antigen-binding portion thereof binds to cell surface human CD3 with a K_{D} of 1×10⁻⁸ M or less, as measured by FACS.

60. The bispecific antibody or the antigen-binding portion thereof of claim 51, wherein the bispecific antibody or the antigen-binding portion thereof is cross-reactive to cynomolgus monkey CD3 and CD20 antigens.

61. An isolated nucleic acid molecule, comprising a nucleic acid sequence encoding the bispecific antibody or the antigen-binding portion thereof as defined in claim 51.

62. A vector comprising the isolated nucleic acid molecule of claim 61.

63. A host cell comprising the vector of claim 62.

64. A pharmaceutical composition comprising the bispecific antibody or the antigen-binding portion thereof as defined in claim 51 and a pharmaceutically acceptable carrier.

65. A method for preparing a bispecific antibody or antigen-binding portion thereof as defined in claim 51, comprising the steps of:
expressing the bispecific antibody or antigen-binding portion thereof in a host cell, wherein the host cell comprises a vector comprising an isolated nucleic acid sequence, wherein the isolated nucleic acid sequence comprises a nucleic acid sequence encoding the bispecific antibody or the antigen-binding portion thereof as defined in claim 51; and
isolating the bispecific antibody or antigen-binding portion thereof from the host cell.

66. A method for inhibiting growth of tumor cells in a subject, comprising administering an effective amount of the bispecific antibody or the antigen-binding portion thereof as defined in claim 51 or the pharmaceutical composition comprising the bispecific antibody or the antigen-binding portion thereof as defined in claim 51 to the subject.

67. A method for reducing tumor cell metastasis in a subject, comprising administering an effective amount of the bispecific antibody or the antigen-binding portion thereof as defined in claim 51 or the pharmaceutical composition comprising the bispecific antibody or the antigen-binding portion thereof as defined in claim 51 to the subject.

68. A method for treating or preventing diseases (such as cancers) comprising proliferative disorders, autoimmune diseases, inflammatory disease or infectious diseases in a subject, comprising administering an effective amount of the bispecific antibody or the antigen-binding portion thereof as defined in claim 51 or the pharmaceutical composition comprising the bispecific antibody or the antigen-binding portion thereof as defined in claim 51 to the subject.

69. The method of claim 68, wherein the cancers comprise B-cell cancers, for example, chronic lymphoid lymphoma (CLL) and non-Hodgkin's lymphoma (NHL).

70. A kit for treating or diagnosing proliferative disorders (such as cancers), autoimmune diseases, inflammatory disease or infectious diseases, comprising a container comprising the bispecific antibody or the antigen-binding portion thereof as defined in claim 51.


SEQUENCE LISTING (42 sequences)

SEQ ID NO: 1 - protein, 10 aa, artificial sequence
  CDR1 in the heavy chain variable region (“VH”) of “anti-CD3 arm”
GFAFTDYYIH

SEQ ID NO: 2 - protein, 17 aa, artificial sequence
  CDR2 in the heavy chain variable region of “anti-CD3 arm”
WISPGNVNTKYNENFKG

SEQ ID NO: 3 - protein, 10 aa, artificial sequence
  CDR3 in the heavy chain variable region of “anti-CD3 arm”
DGYSLYYFDY

SEQ ID NO: 4 - protein, 17 aa, artificial sequence
  CDR1 in the light chain variable region (“VL”) of “anti-CD3 arm”
KSSQSLLNSRTRKNYLA

SEQ ID NO: 5 - protein, 7 aa, artificial sequence
  CDR2 in the light chain variable region of “anti-CD3 arm”
WASTRQS

SEQ ID NO: 6 - protein, 8 aa, artificial sequence
  CDR3 in the light chain variable region of “anti-CD3 arm”
TQSHTLRT

SEQ ID NO: 7 - protein, 10 aa, artificial sequence
  CDR1 in the heavy chain variable region of “anti-CD20 arm”
GFTFNDYAMH

SEQ ID NO: 8 - protein, 17 aa, artificial sequence
  CDR2 in the heavy chain variable region of “anti-CD20 arm”
TISWNSGSIGYADSVKG

SEQ ID NO: 9 - protein, 13 aa, artificial sequence
  CDR3 in the heavy chain variable region of “anti-CD20 arm”
DIQYGNYYYGMDV

SEQ ID NO: 10 - protein, 11 aa, artificial sequence
  CDR1 in the light chain variable region of “anti-CD20 arm”
RASQSVSSYLA

SEQ ID NO: 11 - protein, 7 aa, artificial sequence
  CDR2 in the light chain variable region of “anti-CD20 arm”
DASNRAT

SEQ ID NO: 12 - protein, 9 aa, artificial sequence
  CDR3 in the light chain variable region of “anti-CD20 arm”
QQRSNWPIT

SEQ ID NO: 13 - protein, 119 aa, artificial sequence
  Amino acid sequence of VH of “anti-CD3 arm”
QVQLVQSGAEVKKPGSSVKVSCKASGFAFTDYYIHWVRQAPGQGLEWMGWISPGNVNTKYNENFKGRVTITADKSTSTAYMELSSLRSEDTAVYYCARDGYSLYYFDYWGQGTLVTVSS

SEQ ID NO: 14 - protein, 112 aa, artificial sequence
  Amino acid sequence of VL of “anti-CD3 arm”
DIVMTQSPDSLAVSLGERATINCKSSQSLLNSRTRKNYLAWYQQKPGQPPKLLIYWASTRQSGVPDRFSGSGSGTDFTLTISSLQAEDVAVYYCTQSHTLRTFGGGTKVEIK

SEQ ID NO: 15 - protein, 122 aa, artificial sequence
  Amino acid sequence of VH of “anti-CD20 arm”
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSS

SEQ ID NO: 16 - protein, 107 aa, artificial sequence
  Amino acid sequence of VL of “anti-CD20 arm”
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIK

SEQ ID NO: 17 - DNA, 357 bp, artificial sequence
  DNA sequence encoding VH of “anti-CD3 arm”
caggtgcagcttgtgcagtctggggcagaagtgaagaagcctgggtctagtgtcaaggtgtcatgcaaggctagcgggttcgcctttactgactactacatccactgggtgcggcaggctcccggacaagggttggagtggatgggatggatctccccaggcaatgtcaacacaaagtacaacgagaacttcaaaggccgcgtcaccattaccgccgacaagagcacctccacagcctacatggagctgtccagcctcagaagcgaggacactgccgtctactactgtgccagggatgggtactccctgtattactttgattactggggccagggcacactggtgacagtgagctcc

SEQ ID NO: 18 - DNA, 336 bp, artificial sequence
  DNA sequence encoding VL of “anti-CD3 arm”
gatatcgtgatgacccagagcccagactcccttgctgtctccctcggcgaaagagcaaccatcaactgcaagagctcccaaagcctgctgaactccaggaccaggaagaattacctggcctggtatcagcagaagcccggccagcctcctaagctgctcatctactgggcctccacccggcagtctggggtgcccgatcggtttagtggatctgggagcgggacagacttcacattgacaattagctcactgcaggccgaggacgtggccgtctactactgtactcagagccacactctccgcacattcggcggagggactaaagtggagattaag

SEQ ID NO: 19 - DNA, 366 bp, artificial sequence
  DNA sequence encoding VH of “anti-CD20 arm”
gaggtgcaattggtggagagcggaggagggctcgtgcagcctggaagatctcttaggctgagttgcgctgcatctgggttcacattcaacgactacgccatgcactgggtgaggcaggctcccggcaaagggctggaatgggtgtcaactatctcctggaactccggcagcatcggctacgccgatagcgtcaagggccggtttacaatttcccgcgataacgccaagaagtccctgtacctgcagatgaacagcctgcgggccgaggatactgccctctactactgtgccaaggacattcagtacgggaattactattacgggatggacgtctggggccaggggaccaccgtgacagtcagctcc

SEQ ID NO: 20 - DNA, 321 bp, artificial sequence
  DNA sequence encoding VL of “anti-CD20 arm”
gaaatcgtgctgacccagtccccagcaaccctctccctttctcctggagagagagctaccctcagctgtagggcctcacagtctgtctccagttacctggcttggtaccagcagaaacccgggcaggcccctaggttgctgatctacgacgccagcaatagggccactggcatcccagcccggttttccggaagcggcagcgggacagatttcacactcactattagcagcctggagcccgaggacttcgccgtgtactattgccagcagcggtccaactggcccattacatttggccaagggacacgcctggagattaag

SEQ ID NO: 21 - protein, 25 aa, artificial sequence [sequence omitted]

SEQ ID NO: 22 - protein, 14 aa, artificial sequence
  FW2 in the heavy chain variable region of “anti-CD3 arm”
WVRQAPGQGLEWMG

SEQ ID NO: 23 - protein, 32 aa, artificial sequence
  FW3 in the heavy chain variable region of “anti-CD3 arm”
RVTITADKSTSTAYMELSSLRSEDTAVYYCAR

SEQ ID NO: 24 - protein, 11 aa, artificial sequence
  FW4 in the heavy chain variable region of “anti-CD3 arm”
WGQGTLVTVSS

SEQ ID NO: 25 - protein, 23 aa, artificial sequence
  FW1 in the light chain variable region of “anti-CD3 arm”
DIVMTQSPDSLAVSLGERATINC

SEQ ID NO: 26 - protein, 15 aa, artificial sequence
  FW2 in the light chain variable region of “anti-CD3 arm”
WYQQKPGQPPKLLIY

SEQ ID NO: 27 - protein, 32 aa, artificial sequence
  FW3 in the light chain variable region of “anti-CD3 arm”
GVPDRFSGSGSGTDFTLTISSLQAEDVAVYYC

SEQ ID NO: 28 - protein, 10 aa, artificial sequence
  FW4 in the light chain variable region of “anti-CD3 arm”
FGGGTKVEIK

SEQ ID NO: 29 - protein, 25 aa, artificial sequence [sequence omitted]

SEQ ID NO: 30 - protein, 14 aa, artificial sequence
  FW2 in the heavy chain variable region of “anti-CD20 arm”
WVRQAPGKGLEWVS

SEQ ID NO: 31 - protein, 32 aa, artificial sequence
  FW3 in the heavy chain variable region of “anti-CD20 arm”
RFTISRDNAKKSLYLQMNSLRAEDTALYYCAK

SEQ ID NO: 32 - protein, 11 aa, artificial sequence
  FW4 in the heavy chain variable region of of “anti-CD20 arm”
WGQGTTVTVSS

SEQ ID NO: 33 - protein, 23 aa, artificial sequence
  FW1 in the light chain variable region of “anti-CD20 arm”
EIVLTQSPATLSLSPGERATLSC

SEQ ID NO: 34 - protein, 15 aa, artificial sequence
  FW2 in the light chain variable region of “anti-CD20 arm”
WYQQKPGQAPRLLIY

SEQ ID NO: 35 - protein, 32 aa, artificial sequence
  FW3 in the light chain variable region of “anti-CD20 arm”
GIPARFSGSGSGTDFTLTISSLEPEDFAVYYC

SEQ ID NO: 36 - protein, 10 aa, artificial sequence
  FW4 in the light chain variable region of of “anti-CD20 arm”
FGQGTRLEIK

SEQ ID NO: 37 - protein, 725 aa, artificial sequence
  Full-length amino acid sequence of “anti-CD3 arm”
DIVMTQSPDSLAVSLGERATINCKSSQSLLNSRTRKNYLAWYQQKPGQPPKLLIYWASTRQSGVPDRFSGSGSGTDFTLTISSLQAEDVAVYYCTQSHTLRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSGGGGSGGGGSGGGGSGGGGSQVQLVQSGAEVKKPGSSVKVSCKASGFAFTDYYIHWVRQAPGQGLEWMGWISPGNVNTKYNENFKGRVTITADKSTSTAYMELSSLRSEDTAVYYCARDGYSLYYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 38 - protein, 723 aa, artificial sequence
  Full-length amino acid sequence of “anti-CD20 arm”
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSGGGGSGGGGSGGGGSGGGGSEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 39 - DNA, 2175 bp, artificial sequence
  Nucleotide sequence encoding “anti-CD3 arm”
gatatcgtgatgacccagagcccagactcccttgctgtctccctcggcgaaagagcaaccatcaactgcaagagctcccaaagcctgctgaactccaggaccaggaagaattacctggcctggtatcagcagaagcccggccagcctcctaagctgctcatctactgggcctccacccggcagtctggggtgcccgatcggtttagtggatctgggagcgggacagacttcacattgacaattagctcactgcaggccgaggacgtggccgtctactactgtactcagagccacactctccgcacattcggcggagggactaaagtggagattaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtggcggaggcggcagcggcggagggggatccggaggcggcgggagcggcggcggagggagcggaggaggcgggtccggaggcggcgggagtggaggaggagggtccggcggcggagggagcggaggaggagggagcggcgggggcgggtctggaggaggcgggtccggaggaggcgggtcacaggtgcagcttgtgcagtctggggcagaagtgaagaagcctgggtctagtgtcaaggtgtcatgcaaggctagcgggttcgcctttactgactactacatccactgggtgcggcaggctcccggacaagggttggagtggatgggatggatctccccaggcaatgtcaacacaaagtacaacgagaacttcaaaggccgcgtcaccattaccgccgacaagagcacctccacagcctacatggagctgtccagcctcagaagcgaggacactgccgtctactactgtgccagggatgggtactccctgtattactttgattactggggccagggcacactggtgacagtgagctccgcgtcgaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatgccaggaggagatgaccaagaaccaggtcagcctgtggtgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 40 - DNA, 2169 bp, Artificial Sequence
  Nucleotide sequence encoding “anti-CD3 arm”
gaaatcgtgctgacccagtccccagcaaccctctccctttctcctggagagagagctaccctcagctgtagggcctcacagtctgtctccagttacctggcttggtaccagcagaaacccgggcaggcccctaggttgctgatctacgacgccagcaatagggccactggcatcccagcccggttttccggaagcggcagcgggacagatttcacactcactattagcagcctggagcccgaggacttcgccgtgtactattgccagcagcggtccaactggcccattacatttggccaagggacacgcctggagattaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtggcggaggcggcagcggcggagggggatccggaggcggcgggagcggcggcggagggagcggaggaggcgggtccggaggcggcgggagtggaggaggagggtccggcggcggagggagcggaggaggagggagcggcgggggcgggtctggaggaggcgggtccggaggaggcgggtcagaggtgcaattggtggagagcggaggagggctcgtgcagcctggaagatctcttaggctgagttgcgctgcatctgggttcacattcaacgactacgccatgcactgggtgaggcaggctcccggcaaagggctggaatgggtgtcaactatctcctggaactccggcagcatcggctacgccgatagcgtcaagggccggtttacaatttcccgcgataacgccaagaagtccctgtacctgcagatgaacagcctgcgggccgaggatactgccctctactactgtgccaaggacattcagtacgggaattactattacgggatggacgtctggggccaggggaccaccgtgacagtcagctccgcgtcgaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtgcaccctgcccccatcccaggaggagatgaccaagaaccaggtcagcctgagctgcgcggtcaaaggcttctaccccagcgacatcgccgtggagtgggagagcaatgggcagccggagaacaactacaagaccacgcctcccgtgctggactccgacggctccttcttcctcgttagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 41 - protein, 12 aa, artificial sequence
  Sequence of linker (“hinge sequence”)
ESKYGPPCPPCP

SEQ ID NO: 42 - protein, 217 aa, artificial sequence
  Human IgG4 Fc region
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK